(19) United States
(12) Patent Application Publication
(10) Pub. No.: US 2025/0241582 A1
(43) Pub. Date: Jul. 31, 2025

(54) A METHOD, SYSTEM, DEVICE AND COMPUTER PROGRAM

(71) Applicant: Sony Group Corporation, Tokyo (JP)

(72) Inventors: Ayaka Hirata, Tokyo (JP); David Duffy, London (GB); Christopher Wright, London (GB); Nicholas Walker, London (GB)

(73) Assignee: Sony Group Corporation, Tokyo (JP)

(21) Appl. No.: 18/685,653

(22) PCT Filed: Jun. 21, 2022
(86) PCT No.: PCT/JP2022/024626

§ 371 Date: Feb. 22, 2024

(30) Foreign Application Priority Data
Sep. 10, 2021 (EP) 21196017.4

(51) Int. Cl. A61B 5/00; A61B 5/11; A61B 5/16; G16H 50/20

(52) U.S. Cl. CPC A61B 5/4088; A61B 5/1104; A61B 5/163; A61B 5/165; G16H 50/20

(57) ABSTRACT

The present disclosure relates to a method for generating a parameter related to a psychiatric disorder, comprising: displaying to a user a first image set, the first image set comprising a first image having a first trigger image and a second image different from the first image, such that the first image and the second image are displayed to each eye of the user, detecting a first movement of the user whilst the first image set is displayed; displaying to the user a second image set, the second image set comprising a third image having a second trigger image and a fourth image different from the third image, such that the third image and the fourth image are displayed to each eye of the user; detecting a second movement of the user whilst the second image set is displayed; and generating a parameter related to the psychiatric disorder in the user based on user activity data that is indicative of the first movement of the user and the second movement of the user.

### TECHNICAL FIELD

The present disclosure relates to a method, system, device and computer program for generating a parameter related to a psychiatric disorder.

### BACKGROUND

Many assessments for psychiatric disorder symptoms require the user to participate in a specific test with an expert. Assessment methods can also be invasive and require specific test equipment. As a result, such methods can be inconvenient to implement and uncomfortable for the user, which may reduce the likelihood of users repeatedly undergoing assessments. Some psychiatric disorders, such as those presenting hyperactivity and attention deficit symptoms, are also known to primarily present symptoms in childhood, often before school-age. There is a lack of assessment techniques particularly suitable for children, however.

It is therefore an aim of embodiments of the disclosure to create a method of assessing psychiatric disorder symptoms that is more convenient, comfortable for the user and suitable for children to increase the instances of reliable diagnoses.

### SUMMARY

The present disclosure is defined by the claims.

### BRIEF DESCRIPTION OF DRAWINGS

Non-limiting embodiments and advantages of the present disclosure will be best understood by reference to the following detailed description taken in conjunction with the accompanying drawings.

FIG. 1a shows a visual feature and a test feature which are displayed to a user in embodiments.

FIG. 1b shows how a visual feature within a scene is replaced with a test feature in embodiments.

FIG. 2 is a flow chart of a method 200 according to embodiments.

FIG. 3 is a diagram of a system 300 according to embodiments.

FIG. 4 is a diagram of an information processing device 400 according to embodiments.

FIG. 5 is a flow chart showing the information flow in the system 300 when the system carries out a method 600 in embodiments.

FIG. 6 is a flowchart of the method 600 according to embodiments.

FIG. 7a shows a video game scene that is displayed to the user in embodiments.

FIG. 7b shows how a user response is defined in embodiments.

FIG. 8a depicts a user interface scene that is displayed to the user in embodiments.

FIG. 8b depicts a user interface scene that is displayed to the user in embodiments.

FIG. 8c depicts a user interface scene that is displayed to the user in embodiments.

FIG. 9 depicts another user interface scene that is displayed to the user in embodiments.

FIG. 10 is a flow chart showing how the user response confidence is calculated in embodiments.

FIG. 11 is a flow chart showing a process carried out by a data sufficiency check algorithm in embodiments.

FIG. 12 is a flowchart of a method 1200 according to embodiments.

FIG. 13 is a graph plotting a dominance switch indicator value against delay time in embodiments.

FIG. 14a shows messages displayed on the screen of a personal device in embodiments.

FIG. 14b shows messages displayed on the screen of a personal device in embodiments.

FIG. 15 shows how a notification displayed on a personal device appears in embodiments.

FIG. 16a shows messages displayed on a wrist device in embodiments.

FIG. 16b shows messages displayed on a wrist device in embodiments.

FIG. 17a depicts messages presented to a recipient in embodiments where multiple disorder probabilities are calculated.

FIG. 17b depicts messages presented to a recipient in embodiments where multiple disorder probabilities are calculated.

FIG. 18 is a flowchart of a method 1800 according to embodiments.

FIG. 19 is a diagram of a system 1900 according to embodiments.

FIG. 20 is a diagram of a system 2000 according to embodiments.

Like reference numerals designate identical or corresponding parts throughout the drawings.

### DESCRIPTION OF EMBODIMENTS

Binocular rivalry is a visual perception phenomenon where, when two different images are presented to each eye, a user's perception of which image is being shown will alternate over time. The image that the user perceives as being shown is dependent upon which eye is ‘dominant’ at a particular time. The dominant eye will typically switch between the left eye and the right eye around every 0.5 seconds. This switch will hereafter be referred to as a ‘dominance switch’, and the time period between each switch will be referred to as the ‘inter-dominance period’.

Studies have shown that longer inter-dominance periods, indicated by a user perceiving the image shown to switch at a slower rate, are a predictor of some psychiatric disorders. For example, average inter-dominance periods of patients with attention deficit hyperactivity disorder (ADHD) have been found to be almost twice that of patients without ADHD (approximately 0.4-0.5 seconds compared to around 0.8 seconds). In addition, measurements of a person's inter-dominance periods can be used to assess the severity of ADHD at a particular time.

Further studies have showed that slower inter-dominance periods may be good predictors of other psychiatric disorders such as bipolar disorder, depression, obsessive compulsive disorder (OCD), autism spectrum disorder (ASD) and schizophrenia.

In the present disclosure two images are simultaneously displayed to a user, one image being shown to the right eye and the other image shown to the left eye. Both images depict a scene. For example, a video game scene may comprise a background graphic, a video game character, a series of platforms or the like. Another example is a user interface scene, which may comprise a series of app icons or the like. A scene may also be a blank or transparent image file.

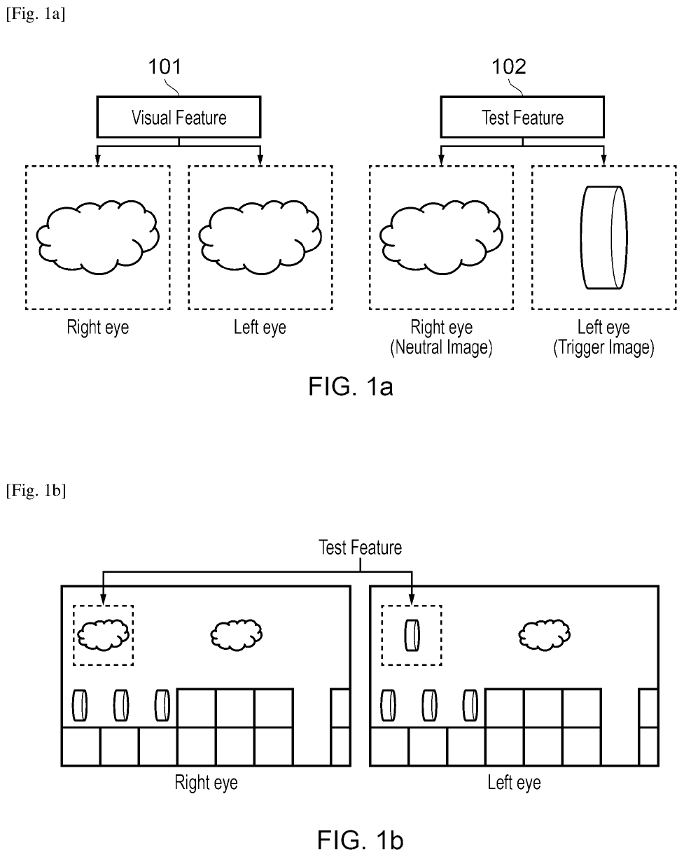

FIG. 1a shows a visual feature 101 and a test feature 102 displayed to a user in embodiments of the present disclosure. A visual feature is an image set comprising two images that are simultaneously displayed to a user, one image being shown to the right eye and the other image being shown to the left eye. A visual feature comprises a part or whole of the entire image shown to each eye, and therefore a visual feature may comprise images that each depict an entire scene, or a particular element within a scene. For example, in some embodiments a visual feature comprises two images depicting a particular section of a video game scene (such as a particular item in the game, or an area of the background) and in other embodiments a visual feature comprises a particular user interface element in a user interface scene (such as a single app icon). In FIG. 1a, visual feature 101 comprises two identical images, both depicting a cloud from a video game scene.

A test feature is a type of visual feature wherein each of its two images are different. In some examples the two images may depict different subjects, whereas in other examples they may depict the same subject from different angles. The two images of a test feature consist of an image configured so as to incite a predetermined response in the user and an image configured so as not to incite a predetermined response in the user. These will be referred to as the ‘trigger image’ and the ‘neutral image’ respectively.

For example, in embodiments where the scene is a video game scene a trigger image may depict a target object for the player to attempt to reach (e.g. a coin to collect), or an object for the player to avoid (e.g. an enemy or trap). Here a neutral image may depict a blank area of background or a section of scenery. In FIG. 1a), test feature 102 comprises an image of a cloud from a video game scene and an image of a coin from the video game scene. In embodiments where the scene is a user interface scene, a trigger image may depict a UI element with a visibly distinct appearance to surrounding UI elements (e.g. a different colour) or a notification (e.g. a red badge on an app icon, a ‘message received’ icon on a status bar, or a pop-up notification itself). Here a neutral image may depict a UI element that is not visibly distinct from surrounding UI elements, or a UI element without a notification (e.g., an app icon with no red notification badge, a status bar with no ‘message received’ icon, or a background UI graphic with no notification). In some embodiments a neutral image consists of no image, for example, a transparent image file with no data contained.

The trigger image is configured to incite a predetermined response in a user to ensure that the user will produce the response whenever they see the trigger image, and therefore that the user will produce the predetermined response whenever the trigger image is shown to the dominant eye of the user. As the typical inter-dominance period of users without a psychiatric disorder is of the order of half a second, it is necessary to configure the trigger image to incite a response in the user as quickly as possible after the trigger image is shown to the dominant eye. This avoids the situation where a user produces a response at a point in time when they no longer see the trigger image, for example if a dominance switch occurs between the user seeing the trigger image and the user producing the response. It is therefore desirable to use a trigger image configured to be as immediately attention-grabbing as possible. Using a trigger image configured to depict a target object or object to avoid in a video game is advantageous because it is likely that the user will be actively looking out for the appearance of such items and therefore be more likely to respond to these quickly. Using a trigger image configured to mimic a notification format familiar to the user (such as a red badge on an app icon) is advantageous because the user will already be accustomed to paying attention to or looking out for such notifications, and therefore be more likely to respond to these quickly. In any embodiment, using a trigger image configured to depict something that is visibly distinct from the surrounding scene (such as being differently-coloured) will also increase the likelihood that the user will respond to it quickly.

FIG. 1b shows how a visual feature 101 within a scene is replaced with a test feature 102 in embodiments. Instead of showing both eyes of the user a cloud in a certain location, the right eye of the user is shown a cloud in that location and the left eye is shown a coin in the same location.

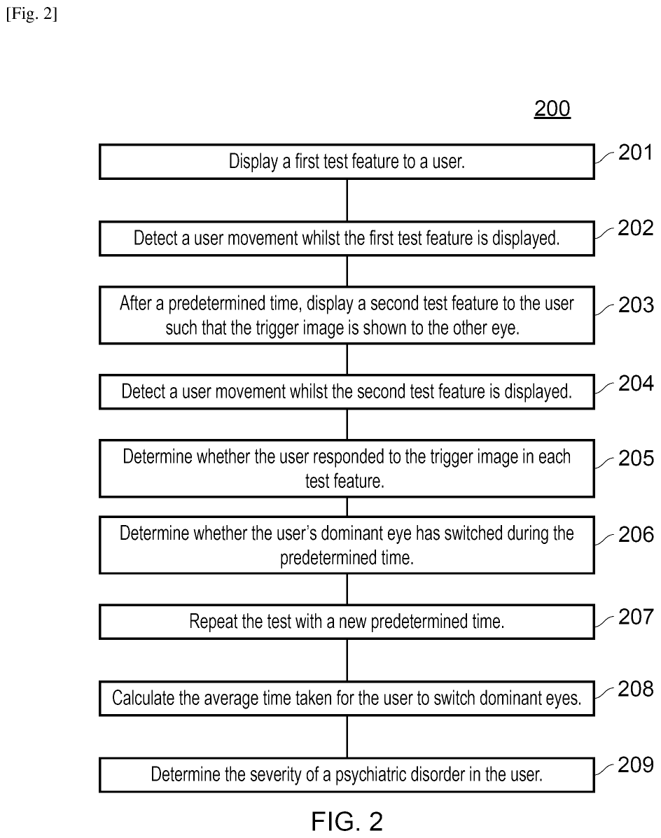

FIG. 2 is a flow chart of a method 200. Method 200 provides a summary of how method 600 and method 1200, which are later described in more detail, are performed in embodiments. In step 201 a first test feature is displayed to a user. In step 202 a movement of the user is detected whilst the first test feature is being displayed. After a predetermined time (the ‘delay time’) has elapsed, a second test feature is displayed to the user in step 203. The first and second test features are configured such that the trigger image of the second test feature and the trigger image of the first test feature are shown to opposite eyes of the user. Thus if the first test feature comprises a trigger image to be shown to the user's left eye, the second test feature comprises a trigger image to be shown to the user's right eye, and vice versa. In step 204 a movement of the user is detected whilst the second test feature is being displayed. In step 205 it is determined whether the user produced a predetermined response when shown each of the first and second test features, in reaction to the trigger image in each test feature. In step 206 it is then determined whether the user's dominant eye has switched during the delay time. The test is repeated such that the first test feature is displayed to the user for a new delay time in step 207. In step 208 the average time taken for the user to switch dominant eyes is calculated. Finally, in step 209 a parameter related to a psychiatric disorder exhibited by the user is determined. In some embodiments this parameter is a calculated severity of the psychiatric disorder. In other embodiments this parameter is a risk of the psychiatric disorder in the user and/or an effect of treatment or medication on the user.

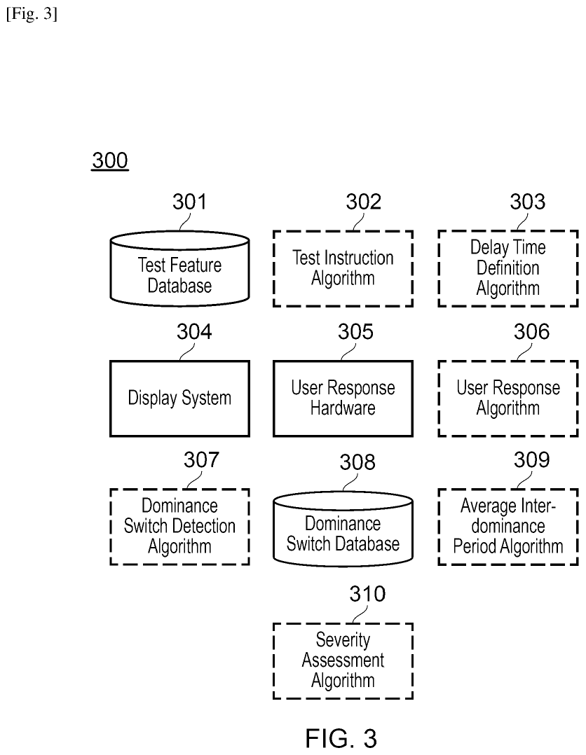

FIG. 3 is a diagram of a system 300 according to embodiments that comprises a test feature database 301, a test instruction algorithm 302, a delay time definition algorithm 303, a display system 304, a user response hardware 305, a user response algorithm 306, a dominance switch detection algorithm 307, a dominance switch database 308, an average inter-dominance period algorithm 309 and an severity assessment algorithm 310.

The test feature database 301 is a database in which preconfigured test features are stored. For each test feature, the database stores information identifying the neutral image and the trigger image as well as a test feature identification tag (the test feature ID) in association with each test feature. In some embodiments, the test feature database 301 additionally stores a visual feature identification tag (the visual feature ID) in association with each test feature.

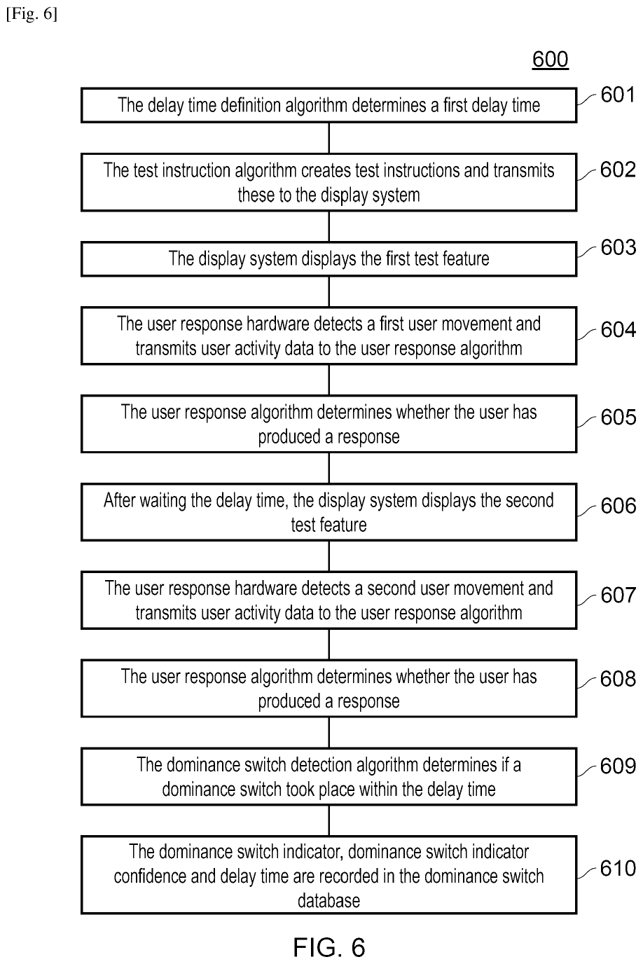

The test instruction algorithm 302 is an algorithm that creates a set of instructions (the test instructions) which define how test features are to be rendered by the display system 304. The test instructions comprise the test feature ID of each test feature to be displayed to the user. In some embodiments the test instructions further comprise a visual feature ID associated with each test feature, an order in which the test features are displayed to the user, and/or a delay time that elapses between displaying the first test feature and displaying the second test feature. In some embodiments the test instruction algorithm 302 creates the test instructions based on information received from the delay time definition algorithm 303, the display system 304 and/or an external database. This is described in more detail with regard to FIG. 6.

The delay time definition algorithm 303 is an algorithm which determines a value for the delay time. This may be achieved via a number of different methods that are described in more detail with reference to FIG. 6.

The display system 304 is a system comprising one or more hardware and/or software components configured to display two images to a user simultaneously. These components may be, for example, the hardware and software drivers included in 3D augmented reality devices, virtual reality devices and other 3D displays known in the art such as head mounted display devices and glasses-type devices. In some embodiments the display system 304 may be configured to simultaneously display two images such that both images are displayed on the same screen. For example, the display system 304 may comprise a screen with two display areas wherein an image displayed on each display area is shown to each eye of the user. When displaying two images to the user simultaneously according to the present disclosure each image is therefore displayed on a different display area.

The user response hardware 305 comprises one or more hardware components configured to detect a movement of the user (a user movement) whilst the display system 304 is displaying a test feature to the user and to transmit user activity data that is indicative of the user movement to the user response algorithm 306. The user response hardware 305 comprises any suitable hardware component known in the art that is capable of being configured in this way. For example, the hardware 305 may include a game controller and measure a user movement using button press detection sensors on the game controller, direction and/or force sensors in a joystick on the game controller or the like. In some embodiments the user response hardware 305 includes a touchscreen and measures a user movement using touchscreen sensors capable of identifying finger taps, swipes or other gestures. In other embodiments, eye tracking hardware for detecting eye movement and/or motion sensing hardware for detecting body movement are included. Eye tracking hardware may be eye-facing cameras for collecting video data or any other sensors capable of providing data for eye-tracking, and may be implemented in embodiments where the display system 304 comprises augmented reality or virtual reality devices. Motion sensing hardware for detecting body movement may include body-facing cameras for body motion assessment, Lidar sensors, light-based motion sensors such as structured-light sensors, accelerometers or the like. In some embodiments the user response hardware 305 comprises motion-sensing hardware that is incorporated in a wearable device, such as accelerometers within a smart watch or other wrist device.

The content and format of the user activity data transmitted by the user response hardware 305 is dependent on the components that make up the hardware 305 in a particular embodiment. For example, a game controller, touchscreen, or other user input device may transmit activity data indicating user actions such as button presses, analogue stick controls, touchscreen interactions or the like. In embodiments where eye-facing cameras are used, eye video data is transmitted. In embodiments where body motion sensing systems are used, motion data is transmitted.

The user response algorithm 306 is an algorithm which analyses the user activity data received from the user response hardware 305 and outputs a user response indicator that indicates whether the user produced a predefined response when the display system 304 was displaying the test feature to the user. In some embodiments the user response algorithm 306 also outputs a user response confidence value that indicates a confidence associated with the user response indicator. This is described in more detail with reference to FIG. 6.

The dominance switch detection algorithm 307 is an algorithm which determines if a user's dominant eye has switched during the delay time. The algorithm produces an output value that indicates whether a dominance switch has occurred (the dominance switch indicator) and in some embodiments calculates a confidence value associated with the dominance switch indicator (the dominance switch indicator confidence). This is described in more detail with reference to FIG. 6.

The dominance switch database 308 is a database which stores dominance switch indicators and corresponding delay times. In some embodiments, the database 308 additionally stores the associated dominance switch indicator confidence values.

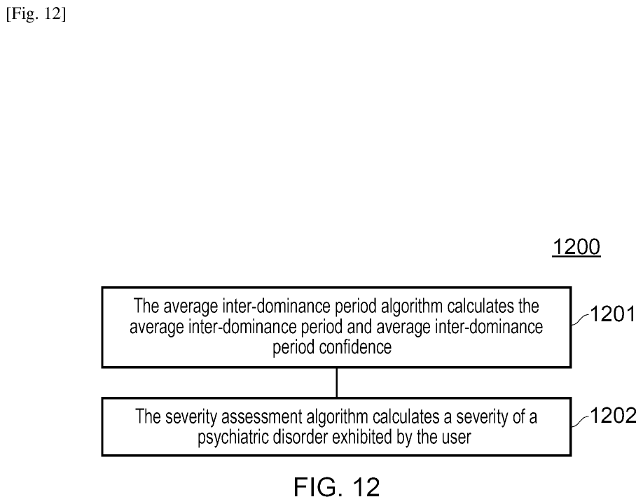

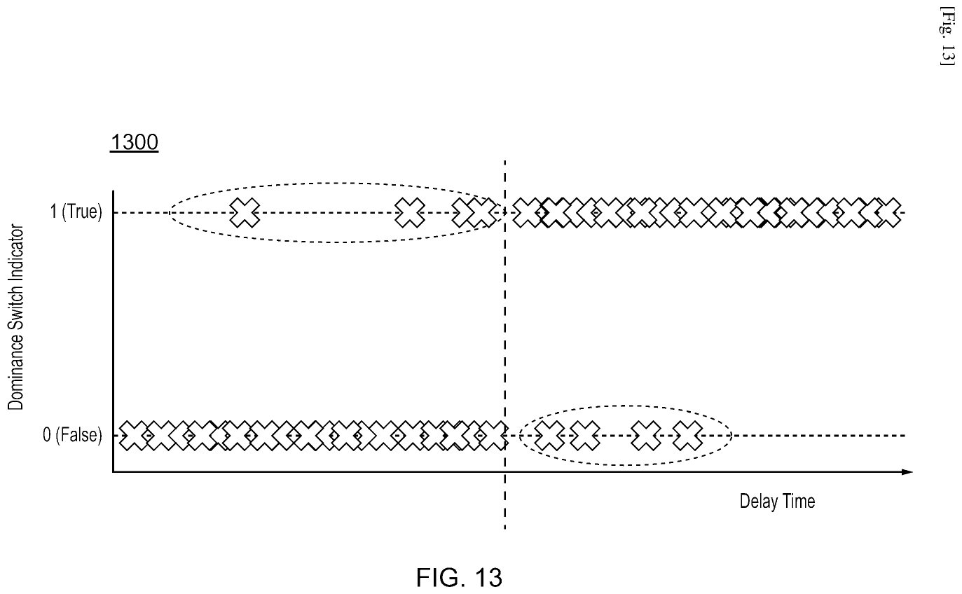

The average inter-dominance period algorithm 309 is an algorithm which uses data from the dominance switch database 308 to calculate an average inter-dominance period for a user. In some embodiments, the average inter-dominance period algorithm 309 additionally calculates a confidence value (the average inter-dominance period confidence) associated with the average inter-dominance period. This is described in more detail with reference to FIGS. 12-13.

The severity assessment algorithm 310 is an algorithm which calculates a parameter related to the psychiatric disorder exhibited by the user. In some embodiments, the severity assessment algorithm 310 additionally outputs a confidence associated with this parameter. In some embodiments this parameter is a severity of the psychiatric disorder. In other embodiments this parameter is the risk of the psychiatric disorder in the user and/or the effectiveness of treatment or medication on the user. This is described in more detail with reference to FIG. 12.

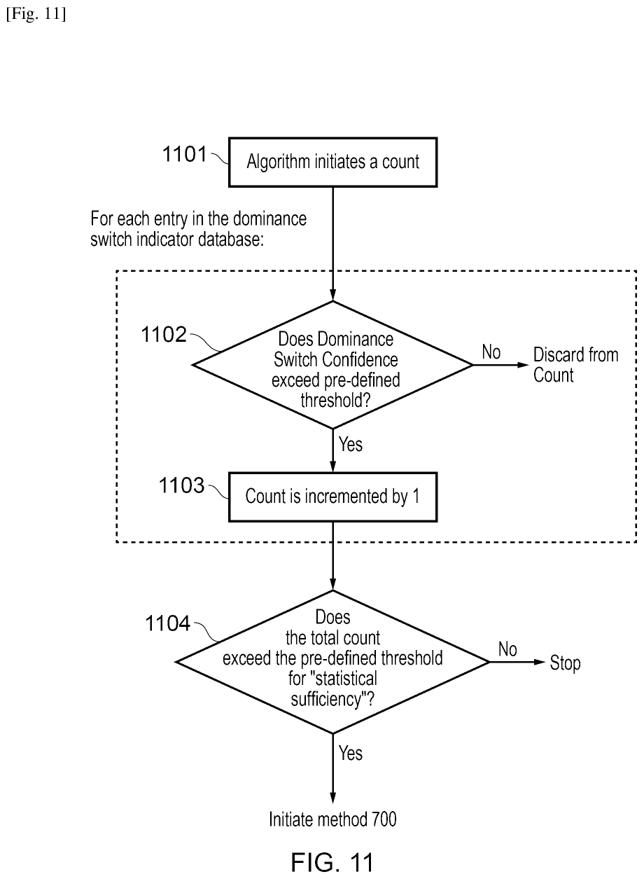

In further embodiments, the system comprises a data sufficiency check algorithm (not shown) which determines whether the number of data entries in the dominance switch database 308 has reached a predetermined threshold. In these embodiments, the average inter-dominance period algorithm 309 will only be run when the number of entries reaches this threshold. This is described in more detail with reference to FIG. 11.

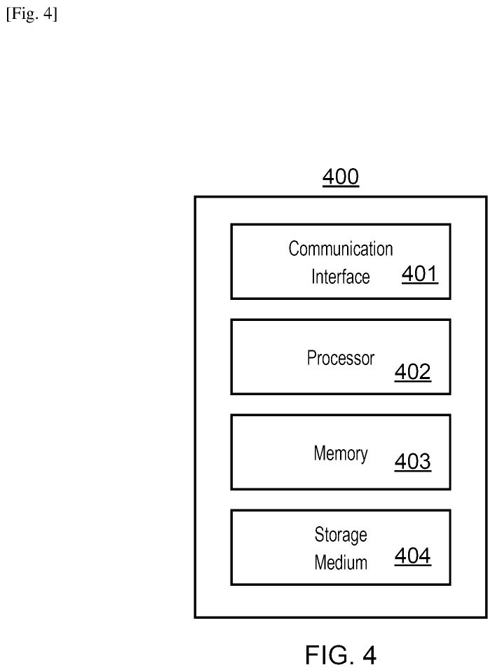

In some embodiments the components of system 300 are located within the same device. Such a device may comprise the display system 304, the user response hardware 305 and an information processing device. FIG. 4 is a diagram of an information processing device 400 according to embodiments. In FIG. 4, the information processing device 400 comprises a communication interface 401 for sending electronic information to and/or receiving electronic information from different components of the system 300, a processor 402 for processing electronic instructions, a memory 403 for storing the electronic instructions to be processed and input and output data associated with the electronic instructions, and a storage medium 404 (e.g. in the form of a hard disk drive, solid state drive, tape drive or the like) for long term storage of electronic information. The processor 402 controls the operation of each of the communication interface 401, memory 403 and storage medium 404.

Each of the algorithms 302, 303, 306, 307, 309 and 310 may be stored on the storage medium 404 as computer readable instructions, which when executed control the processor 402 to implement methods according to embodiments of the present invention. The storage medium 404 may include the test feature database 301 and/or the dominance switch database 308. For example, in embodiments where all the components of system 300 are located within the same device the algorithms 302, 303, 306, 307, 309 and 310 are all stored on the storage medium 404 as described and storage medium 404 includes both the test feature database 301 and the dominance switch database 308.

Each of the communication interface 401, processor 402 and memory 403 are implemented using appropriate circuitry, for example. The circuitry may be embodied as solid state circuitry which may be controlled by software or may be an Application Specific Integrated Circuit. Such software comprises computer readable instructions, which when loaded onto a computer or circuitry, configures the computer (or circuitry) to perform methods according to embodiments. The software is stored on the storage medium 404.

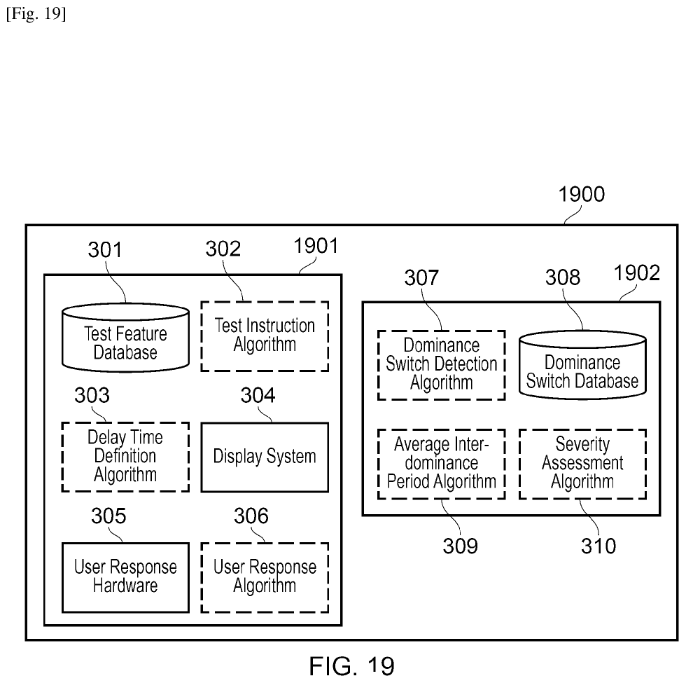

In other embodiments the components of system 300 are located in multiple devices, where each device may comprise an information processing device such as the information processing device 400. In some embodiments one or more components are located in cloud storage. For example, in embodiments the test feature database 301, delay time definition algorithm 303, test instruction algorithm 302 and display system 304 are located in a single device whilst the user response hardware 305 and user response algorithm 306 are within a second device and the dominance switch detection algorithm 307, the dominance switch database 308, the average inter-dominance period algorithm 309 and the severity assessment algorithm 310 are located in cloud storage. Another possible configuration in embodiments is described later with regard to FIG. 19.

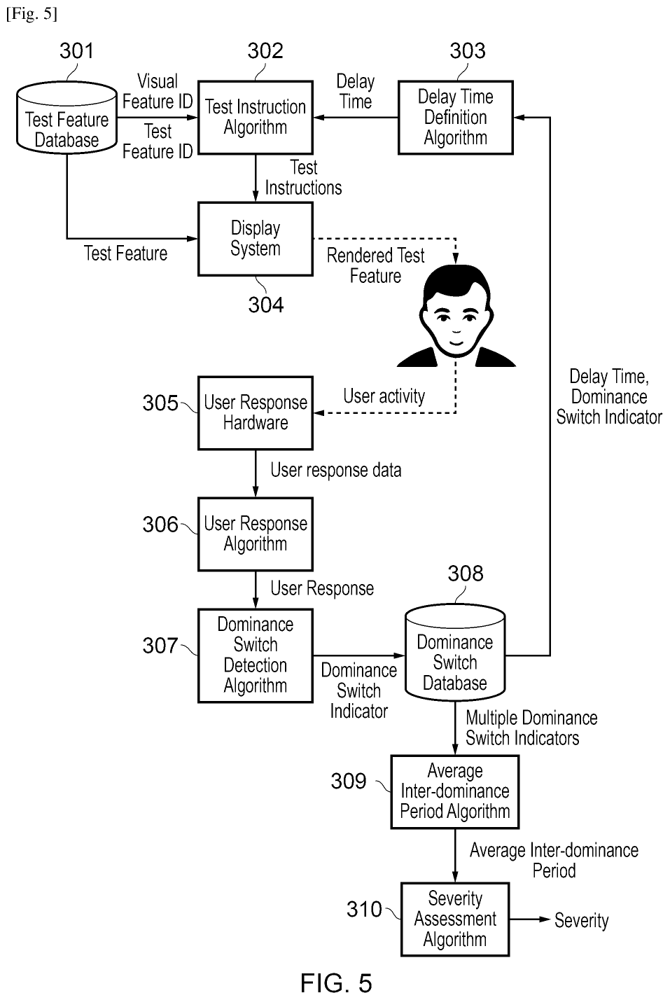

FIG. 5 is a flow chart showing the information flow in the system 300 when the system 300 carries out a method 600 described below.

FIG. 6 shows a method 600 according to embodiments in which the system 300 performs a test to determine a single dominance switch indicator, dominance switch indicator confidence and delay time.

In step 601 the delay time definition algorithm 303 determines a delay time. If the algorithm has not previously determined a delay time and there are therefore no previous delay time values stored in the dominance switch database 308 (i.e. the method 600 is being carried out for the first time), the delay time is determined by choosing a value at random from a predetermined range of values. For example, this range may be Oms to 1200 ms when the severity of ADHD is being assessed.

If the delay time definition algorithm 303 has previously determined a delay time, the delay time is determined based on information received from the dominance switch database 308. For example, in some embodiments the algorithm retrieves the most recently recorded delay time and dominance switch indicator from the dominance switch database 308 and determines the new delay time based on whether a dominance switch occurred within the most recent previous delay time. If the dominance switch indicator indicates that a dominance switch occurred within the most recent previous delay time, the new delay time is determined by subtracting a predetermined time value (e.g. 10 ms) from the most recent previous delay time. If the dominance switch indicator indicates that a dominance switch did not occur within the most recent previous delay time, the new delay time is determined by adding a predetermined time value to the most recent previous delay time. This allows the determined delay times of successive tests to become iteratively closer to the value of the user's average inter-dominance period, since if the most recent previous delay time was longer than the average inter-dominance period the delay time will be reduced and if the most recent previous delay time was shorter than the average inter-dominance period the delay time will be increased. However, the present disclosure is not limited to this and the delay time definition algorithm 303 may determine the delay time by other means (e.g. by choosing a single predetermined value), whether or not the method 600 has previously been carried out.

In some embodiments the delay time is be determined in such a way that it does not exceed a value that is double the average inter-dominance period of a person without a psychiatric disorder (or a value that is within a predetermined range of this time). This decreases the likelihood of a scenario in which a dominance switch occurs more than once during the delay time, which could result in an inaccurate dominance switch indicator being calculated later in step 609.

In step 602 the test instruction algorithm 302 creates test instructions and transmits these to the display system 304. The test instructions comprise the test feature ID of the first and second test features to be displayed to the user, one or more visual feature IDs associated with each of the two test features, the order in which the test features are to be displayed to the user, and the determined delay time.

The visual IDs associated with each test feature are the visual IDs for the visual features that the first and second test features will replace. For example, in FIG. 1b) the visual feature 101 is replaced with test feature 102. The visual ID for visual feature 101 would therefore be stored in association with the test feature 102 in the test feature database 301. The test instruction algorithm 302 first determines the visual IDs for the visual features that the test features will replace, then retrieves associated test feature IDs from the test feature database 301.

In some embodiments the test instruction algorithm 302 determines the visual IDs associated with each test feature by selecting from a list received from the display system 304 that contains the visual IDs for visual features which are about to be displayed. This method is beneficial in embodiments where the visual elements displayed to the user change over time, for example in a video game scene in which the background changes as the player character moves around in the game. In embodiments where the scene does not change over time (for example a user-interface scene containing user interface elements such as app icons, standard home screen buttons or the like), the visual IDs of these visual elements may already be known and so the test instruction algorithm 302 may select the visual IDs from a list.

The test instruction algorithm 302 then retrieves associated test feature IDs from the test feature database 301. When more than one test feature is associated with a single visual feature and/or more than one visual feature is associated with a single test feature, the test instruction algorithm 302 retrieves test features based on predetermined selection criteria. For example, the selection criteria may prioritise choosing test features which are associated with the greatest number of visual IDs to replace.

In some embodiments the test instruction algorithm 302 determines the order in which the first test feature and the second test feature are to be displayed to the user (and therefore which eye of the user is to be first shown a trigger image) by choosing the order at random, or from a predetermined list. In certain embodiments in which the method 600 has been carried out previously, the test instruction algorithm 302 determines the test feature order based on a previous test feature order. For example, the order may be determined such that the eye of the user that is first shown a trigger image alternates in successive tests, or such that the number of tests in which the user's right eye is first shown a trigger image and the number of tests in which the user's left eye is first shown a trigger image lie within a predetermined threshold of each other for a certain number of tests. In another example, the test feature order may be determined based on the delay time, a previous delay time and a previous test feature order. However, the present disclosure is not limited to this and the test instruction algorithm 302 may determine the order in which the first test feature and the second test feature are to be displayed to the user by any suitable means.

In step 603 the display system 304 displays the first test feature to the user according to the test instructions received from the test instruction algorithm 302. The display system 304 first displays visual features to the user that are not test features. For example, a video game scene or a user interface scene may be displayed to the user. The display system 304 then retrieves the first test feature from the test feature database 301 by selecting the test feature that corresponds to the first test feature ID in the test instructions, identifies a visual feature in the scene that corresponds to a visual feature ID associated with the first test feature ID in the test instructions, and replaces the display of this visual feature with the display of the first test feature.

In step 604 the user response hardware 305 detects a first user movement and transmits user activity data to the user response algorithm 306. This may be carried out in a number of different ways depending on the nature of the user response hardware 305 in a particular embodiment, as described above with reference to FIG. 3.

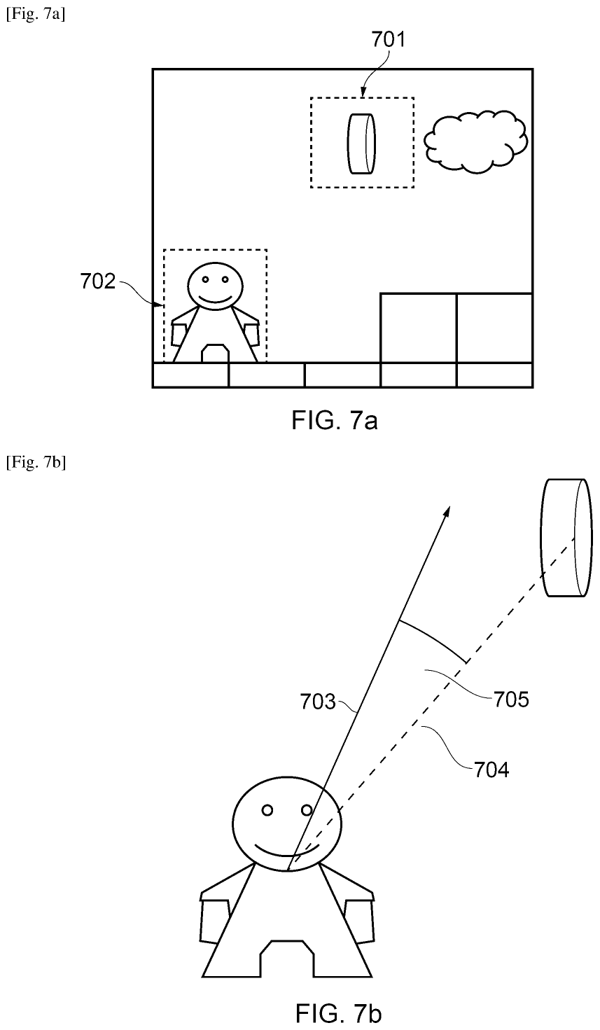

In step 606 the user response algorithm 306 determines whether the user has produced a response during the first user movement. The user activity data received from the user response hardware 305 is analysed to determine whether all or part of the first user movement meets a set of predetermined requirements which define a particular ‘user response’. The nature of this predefined response and the requirements that define it may be selected based on the user response hardware 305, the user movement, the configuration of the test feature and the scene being shown to the user in a particular embodiment. Example embodiments are described in more detail with reference to FIGS. 7-10. The user response algorithm 306 then outputs a user response indicator (‘user response A’) that indicates whether or not the user has produced a response during the first movement, as well as a user response confidence value (‘user response A confidence’) that indicates a confidence associated with user response A. The calculation of this confidence is dependent on the nature of the response and the requirements that define it. Again, example embodiments are described with reference to FIGS. 7-10.

In step 606, the display system 304 finishes displaying the first test feature after the first test feature has been displayed for a time equal to the delay time defined by the delay time definition algorithm 303 in step 601. The display system 304 then displays the second test feature according to the test instructions received from the test instruction algorithm 302. This process is performed in the same way as the display of the first test feature according to the test instructions in step 603, and therefore will not be repeated here.

In step 607 the user response hardware 305 detects a second user movement in the same way as the first user movement was detected in step 604 and again transmits user activity data to the user response algorithm 306. In step 608 the user response algorithm 306 then determines whether the user has produced a response during the second user movement in the same way as it determined whether a response was produced during the first user movement in step 606. The user response algorithm 306 outputs a user response indicator (‘user response B’) that indicates whether or not the user produced a response during the second movement, as well as a user response confidence value (‘user response B confidence’) indicating a confidence associated with user response B.

In step 609 the dominance switch detection algorithm 307 receives the user response A, user response A confidence, user response B and user response B confidence from the user response algorithm 306. The dominance switch detection algorithm 307 then determines whether or not a dominance switch occurred while the first test feature was displayed to the user for the delay time, and outputs a dominance switch indicator that indicates the result of this determination. A dominance switch indicator confidence is also calculated, indicating a confidence associated with the dominance switch indicator value.

The dominance switch detection algorithm 307 determines whether or not a dominance switch has occurred by comparing the user response A and user response B received from the user response algorithm 306. If user response A and user response B indicate identical results (i.e. both indicate that a response was produced or both indicate that a response was not produced), it is determined that a dominance switch occurred. If user response A and user response B indicate different results (i.e. only one indicates that a response was produced), it is determined that a dominance switch did not occur. The dominance switch detection algorithm 307 calculates the dominance switch indicator confidence based on the user response A confidence and the user response B confidence received from the user response algorithm 306. This may be achieved using any suitable mathematical calculation for combining two confidence values that is known in the art.

Finally, in step 610 the dominance switch indicator, dominance switch indicator confidence and delay time are stored in the dominance switch database 308. The system 300 can then, in a method 1200 that is described later, use these values to calculate an average inter-dominance period of the user and then determine a parameter related to a psychiatric disorder exhibited by the user.

FIGS. 7-10 show how the user response algorithm 306 determines a user response indicator and user response indicator confidence in certain embodiments.

FIG. 7a shows how a video game scene is displayed to the user in an embodiment. The test feature 701 is a coin to collect in the video game and the user response hardware 305 comprises a game controller with a joystick. The display system 304 and user response hardware 305 are configured such that moving the joystick in a particular direction causes a character 702 in the scene to move in a corresponding direction in the game. In this embodiment the user movement comprises the user moving the joystick to direct the character to move in the game. Vector 703 shown in FIG. 1b represents the projected direction of movement of the character 702.

Here, the user response is defined as a movement of the joystick such that the vector 703 matches the direction of the test feature 701 from the character 702 (vector 704) with an associated confidence that exceeds a certain threshold. This can be seen in FIG. 7b. In this embodiment the user response confidence is based on the error 705 of the vector 704, this being the magnitude of the angular difference between vector 703 and vector 704. The user response confidence is also based on the magnitude of the vector 704, which corresponds to the speed of the joystick movement. The confidence threshold that defines the user response may be based on whether the error 705 is less than a threshold value and/or whether the magnitude of the vector 704 is greater than a threshold value. However, the present disclosure is not limited to this. In other embodiments the user response confidence may be based on each of these factors individually, and/or on other appropriate factors, and the confidence threshold may be calculated in any appropriate manner. For example, the user response confidence and associated confidence threshold may be based on the force applied to the joystick by the user.

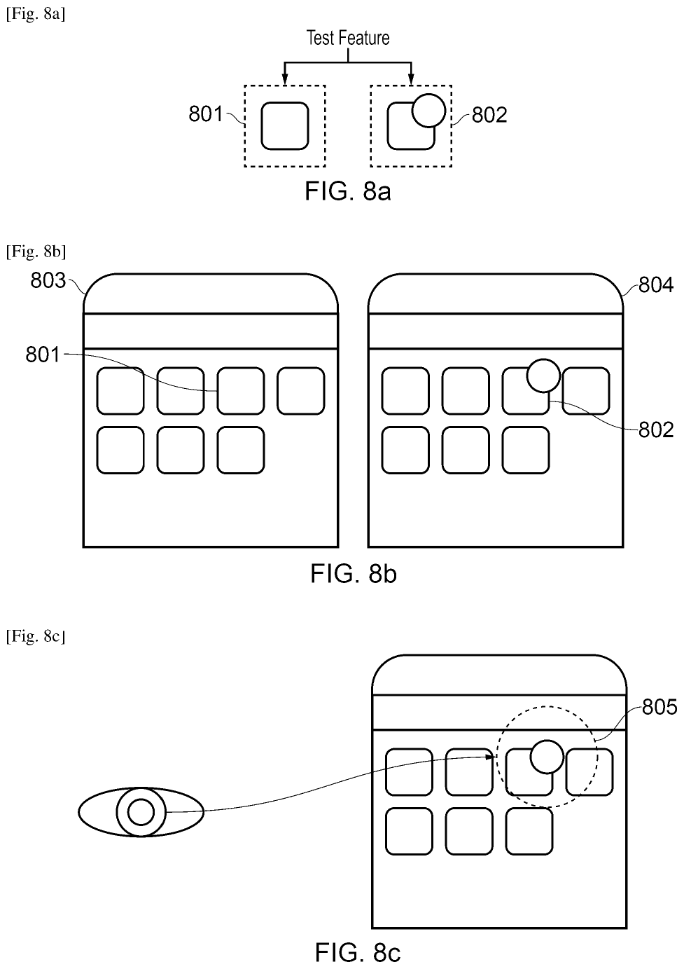

FIG. 8 depicts a user interface scene comprising several app icons that is displayed to the user in an embodiment. In FIG. 8a, a test feature is shown with a neutral image 801 depicting an app icon and a trigger image 802 depicting the same app icon with a red notification badge. FIG. 8b shows the two different images displayed to the user when the test feature is being displayed. The left image 803 comprises the neutral image 801 and the right image 804 comprises the trigger image 802.

In embodiments, the user response hardware 305 comprises eye tracking hardware such as one or more eye-facing cameras and is configured to identify a region of the scene that the eyes of the user are directed to look at (the fixation location). The user movement is the movement of the user's eyes to look at the fixation location. In FIG. 8c the user's right eye is directed towards the fixation location 805 in the right image 804. The fixation location 805 encompasses the entirety of the trigger image 802 and is centred on the red notification badge of the app icon.

Here, the user response is defined as the movement of the user's eyes to look at a fixation location 805 with an associated confidence that exceeds a certain confidence threshold. In this embodiment the user response confidence is based on the distance between the centre of the fixation location 805 and the centre of the trigger image 802. The user response confidence is also based on the time interval between when the display system 304 first starts to display the test feature to the user and when the user's eyes are moved to look at the fixation location 805, and the duration for which the user looks at the fixation location 805. The confidence threshold that defines the user response may be based on whether the distance between the centre of the fixation location 805 and the centre of the trigger image 802 is less than a threshold value, whether the time interval between the first display of the test feature to the user and when the user's eyes were moved to look at the fixation location 805 is less than a threshold value, and/or whether the duration for which the user looks at the fixation location 805 is greater than a threshold value.

However, the present disclosure is not limited to this. In other embodiments the user response confidence is based on each of these factors individually and/or on other appropriate factors, and the confidence threshold is calculated in any appropriate manner. For example, in other embodiments where the user response hardware 305 comprises eye tracking hardware the user response confidence may additionally or alternatively be based on the distance between the centre of the fixation location and a particular region of the test feature (such as the region encompassing the red notification badge). In another example, the confidence threshold may be based on whether the fixation location encompasses the test feature (as seen in FIG. 8c).

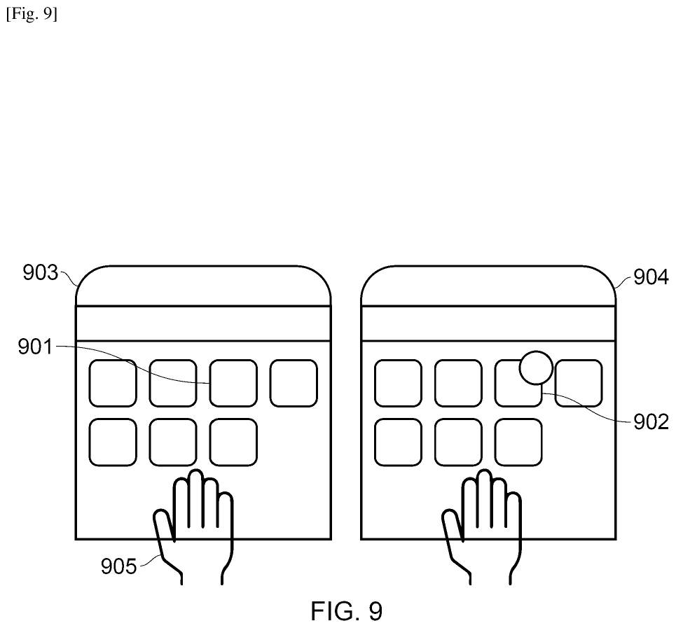

FIG. 9 depicts a user interface scene comprising several app icons that is displayed to the user in another embodiment. The user interface scene is similar to the scene in FIG. 8b but differs in that a user representation is included. FIG. 9 shows the two different images displayed to the user when the test feature is being displayed. The left image 903 comprises the neutral image 901 and the right image 904 comprises the trigger image 902. A user representation 905 in the shape of a hand is included.

In this embodiment the user response hardware 305 comprises motion sensing hardware such as a body-facing camera that is configured to detect body movement of the user, specifically hand movement. The display system 304 and user response hardware 305 are configured such that when the user's physical hand is moved, the user representation 905 is directed to move within the scene in a corresponding direction. In this embodiment the user movement comprises the movement of the user's physical hand to direct the user representation to move in the scene.

Here, the user response is defined as the movement of the user's hand such that the projected direction of movement of the user representation 905 matches the direction of the test feature 901 from the user representation 905 with an associated confidence that exceeds a certain threshold. In this embodiment the user response confidence is based on the magnitude of the angular difference between the projected direction of movement of the user representation 905 and the direction of the test feature 901 from the user representation 905. The user response confidence is also based on the acceleration of the user representation 905, which corresponds to the detected acceleration of the user's physical hand. In some embodiments the confidence threshold that defines the user response is based upon whether the magnitude of the angular difference between the projected direction of movement of the user representation 905 and the direction of the test feature 901 from the user representation 905 is less than a threshold value and/or whether the acceleration of the user representation 905 is greater than a threshold value. However, the present disclosure is not limited to this. In other embodiments the user response confidence may be based on each of these factors individually and/or on other appropriate factors, and the confidence threshold may be calculated in any appropriate manner.

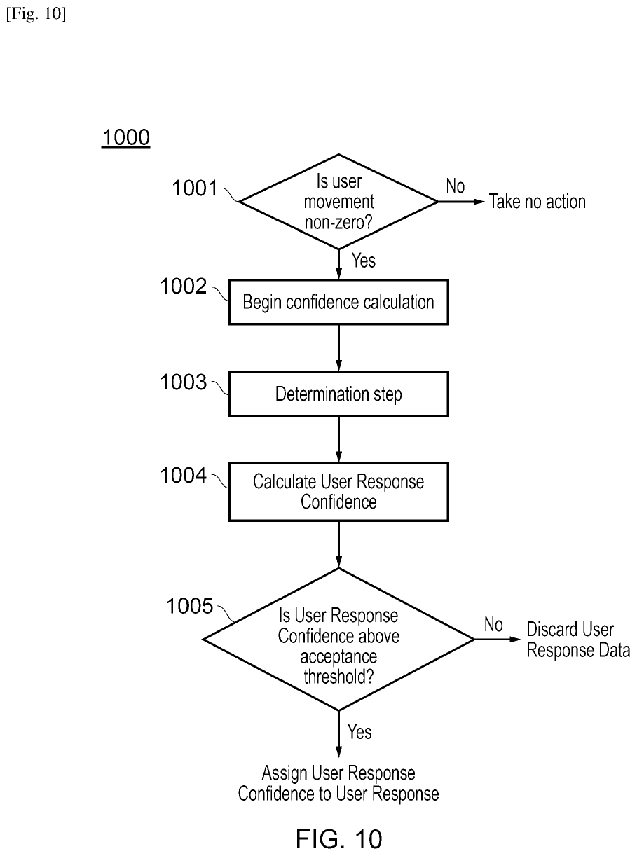

FIG. 10 is a flow chart showing how the user response confidence is calculated in the embodiments depicted in FIGS. 7-9.

In step 1001 it is first determined whether the user movement is non-zero. If the user movement is zero, the process ends. If the user movement is determined to be nonzero, the process moves to step 1002 in which the user response confidence calculation is initiated. In step 1003, a number of determinations are made.

In the embodiment depicted in FIG. 7, this step comprises determining the magnitude of vector 704 and the error 705. In the embodiment depicted in FIG. 8 this step comprises determining the distance between the centre of the fixation location 805 and the centre of the trigger image 802, the time interval between when the display system 304 started to display the test feature to the user and when the user's eyes were moved to look at the fixation location 805, and the duration for which the user looked at the fixation location 805. In the embodiment depicted in FIG. 9, this step comprises determining the magnitude of the angular difference between the projected direction of movement of the user representation 905 and the direction of the test feature 901 from the user representation 905 and the acceleration of the user representation 905. In step 1004 the user response confidence is calculated based on the result of these determinations. Finally, in step 1005 it is determined whether the user response confidence exceeds the predetermined confidence threshold. If the confidence is determined to be below this threshold, the user response algorithm 306 outputs a user response indicator indicating that the user has not produced the predefined response during the user movement. If the confidence is determined to be above this threshold, the user response algorithm 306 outputs a user response indicator indicating that the user has produced the response. In either scenario the associated user response confidence value is also output.

After method 600 has been carried out, in some embodiments the system 300 immediately starts method 1200. This will be described in more detail with reference to FIG. 12. However, in other embodiments method 600 is repeated a number of times before method 1200 is started. As described with regard to step 601, in some embodiments the delay time is determined such that a range of delay times is used for multiple repeats of method 600. Therefore a number of dominance switch indicator values with different associated delay times are recorded in the dominance switch database 308. Method 1200 involves calculating an average inter-dominance period value based on the distribution of dominance switch indicator values recorded in the dominance switch database 308 with delay time. This result is then used to calculate a parameter related to a psychiatric disorder exhibited by the user, such as a value of severity. The calculation of the average inter-dominance period (and consequently the parameter related to the psychiatric disorder in method 1200) therefore becomes more reliable as the method 600 is repeated.

In some embodiments, the system 300 will additionally include a data sufficiency check algorithm which determines whether a number of data entries in the dominance switch database 308 has reached a predetermined ‘sufficiency threshold’. This sufficiency threshold will be a number of entries considered to ensure the average inter-dominance period calculation is within a certain desired level of reliability (e.g. 100 or 1000 entries). If it is determined that the number of data entries does not exceed the sufficiency threshold, the system 300 will not begin method 1200. For example, the system 300 may be configured to either stop performing tests or to continue repeating method 600 until the number of data entries exceeds the sufficiency threshold. If it is determined that the number of data entries does exceed the sufficiency threshold, the system 300 will stop repeating method 600 and begin carrying out method 1200.

The data sufficiency check algorithm performs a count of the number of entries in the dominance switch database 308. In some embodiments the algorithm is configured to perform this count once per predefined time period (e.g. once per day). However, the present disclosure is not limited to this and the data sufficiency check algorithm may be configured to perform this count at a time according to any suitable criteria. For example, the count may be initiated after every test (e.g. immediately after step 610 for every repeat of method 600) or whenever a certain condition is met (e.g. when the number of tests is a multiple of 10).

In some embodiments the data sufficiency check algorithm performs a count of the total number of dominance switch indicator entries. In other embodiments the data sufficiency check algorithm performs a count of the number of dominance switch indicator entries for which the dominance switch indicator confidence exceeds a predetermined confidence threshold. FIG. 11 is a flow chart showing a process carried out by the data sufficiency check algorithm in such embodiments. In step 1101 a count is initiated. The data sufficiency check algorithm then carries out steps 1102 and 1103 for each entry in the dominance switch database 308. In step 1102 it is determined whether the dominance switch indicator confidence exceeds the confidence threshold. If it does, the count is increased by 1 in step 1103. If it does not, the count is not increased. After steps 1102 and 1103 have been carried out for every entry in the dominance switch database 308, step 1104 is initiated in which it is determined whether the total count exceeds the sufficiency threshold. If it does, the system 300 will begin carrying out method 1200.

FIG. 12 is a flow chart showing a method 1200 according to embodiments in which the system 300 calculates the average inter-dominance period of the user and a parameter related to a psychiatric disorder exhibited by the user.

In step 1201 the average inter-dominance period algorithm 309 retrieves data from the dominance switch database 308 that was recorded in each test (each repeat of the method 600) described with regard to FIG. 6. Each data entry will comprise a dominance switch indicator, dominance switch indicator confidence and delay time corresponding to a particular test.

In some embodiments, the average inter-dominance period algorithm 309 retrieves data from the dominance switch database 308 according to specific requirements. For example, the average inter-dominance period algorithm 309 may retrieve data from a certain number of the data entries in an order corresponding to how recent the entry was recorded, or retrieve only data recorded within a specific time period (e.g. the previous month from the time of retrieval). In another example, the average inter-dominance period algorithm 309 may retrieve data collected at specific times or events, such as all data recorded at a particular date and time (e.g. every Monday between 1 pm and 2 pm), or all data recorded at times when it is identified that the user is performing a particular activity (e.g. during a lesson at school). This allows an average inter-dominance period (and therefore a parameter related to a psychiatric disorder) to be calculated for the user at specific times or during specific activities. The ability to examine a parameter such as the severity of a psychiatric disorder exhibited by the user during specific times or during specific activities can be desirable when carrying out diagnosis.

In some embodiments the average inter-dominance period algorithm 309 only retrieves data from entries in which the dominance switch indicator confidence exceeds a predetermined confidence threshold, or retrieves data from a predetermined number of data entries in an order corresponding to the relative magnitudes of the dominance switch indicator confidences. The results from calculating an average inter-dominance period (and/or a parameter related to a psychiatric disorder) using only a subset of the available data can therefore be compared with the results from the same calculation when using all the available data. The reliability of the results from the calculation using all the available data can therefore be evaluated.

The average inter-dominance period algorithm 309 then calculates an average inter-dominance period of the user by determining a delay time value that minimises the number of ‘incorrect’ dominance switch indicators when all retrieved data are considered. The average inter-dominance period confidence is also calculated using the values of dominance switch indicator confidence retrieved from the dominance switch database 308. These calculations will be described in more detail with reference to FIG. 13.

In step 1202 the severity assessment algorithm 310 calculates a parameter related to a psychiatric disorder exhibited by the user. In some embodiments this parameter is the severity of the psychiatric disorder. In one embodiment this is achieved by comparing the average inter-dominance period to one or more thresholds that indicate relative levels of severity. For example, when calculating a severity of ADHD exhibited by the user it may be determined that if the average inter-dominance period exceeds 600 ms within a certain confidence the severity is ‘low’, whereas if the average inter-dominance period does not exceed this threshold the severity is ‘high’. In other embodiments the severity assessment algorithm 310 calculates a severity with a value ranging from 0.0 to 1.0, where 0.0 represents low severity and 1.0 represents high severity. For example, the average inter-dominance period may be used as an input to a pre-defined sigmoid function that returns 0 for a value of average inter-dominance period that is typical in people without a psychiatric disorder and 1 for a value of average inter-dominance period that is typical in people with a psychiatric disorder. However, the present disclosure is not limited to this and in other embodiments the severity assessment algorithm 310 may calculate the severity using any suitable method.

In some embodiments, the parameter related to a psychiatric disorder is the user's risk of the psychiatric disorder. This is a likelihood that the user has the disorder, calculated based on the average inter-dominance period of the user. The risk may be calculated using any suitable means, for example by comparing known average inter-dominance period values for people with and without the psychiatric disorder with the calculated average inter-dominance period of the user.

In further embodiments, the parameter related to a psychiatric disorder is an effectiveness of treatment or medication in the user. This is calculated based on one or more values of the user's average inter-dominance period as well as additional information, such as the user's medication history. This additional information may have been provided by a doctor or the user. In other embodiments the severity assessment algorithm 310 is preconfigured with additional information or receives additional information from another component. The medication effectiveness may be calculated using any suitable means, for example based on a rate of change of average inter-dominance period during certain time periods immediately following a medication dose.

In some embodiments, a confidence associated with the parameter (e.g. the severity confidence) is also calculated using the average inter-dominance period confidence. This may be achieved using any suitable mathematical calculation for combining two confidence values that is known in the art. However, the present disclosure is not limited to this and the severity assessment algorithm 310 may additionally or alternatively calculate this confidence using other factors, depending on the method used to calculate the parameter.

FIG. 13 is a graph plotting dominance switch indicator value against delay time for a number of retrieved data entries. As a dominance switch indicator indicates a binary result of whether or not a dominance switch has occurred, it can take one of two values: a value indicating that a switch has occurred (‘true’) and a value indicating that a switch has not occurred (‘false’). Here, ‘true’ and ‘false’ are represented by the values 1 and 0 respectively. Each cross represents a value of dominance switch indicator for a particular delay time. Each cross that is circled by a dotted line represents a value that is determined to be an ‘incorrect’ value in relation to a particular delay time indicated by the vertical dotted line.

In embodiments, the average inter-dominance period algorithm 309 determines a delay time value that minimises the number of incorrect dominance switch indicators using an iterative method. In this method, a delay time is selected from within the range of delay times retrieved from the dominance switch database 308. The average inter-dominance period algorithm 309 then performs a count of the number of values which are ‘incorrect’ in relation to this selected delay time. These values are defined as the ‘true’ value dominance switch indicators with corresponding delay times shorter than the selected delay time and the ‘false’ value dominance switch indicators with corresponding delay times longer than the selected delay time. The average inter-dominance period algorithm 309 then repeats this process for a number of different selected delay times, performing a count of the number of values which are ‘incorrect’ in relation to each selected delay time.

In some embodiments the delay times for which the average inter-dominance period algorithm 309 performs a count are selected based on certain criteria. For example, in certain embodiments these delay times are selected such that the delay time values are spread evenly across the total range of delay times retrieved from the dominance switch database 308. In other embodiments they are selected such that each selected delay time is separated from the previous by the same time interval. This ensures that the total range of delay times of the retrieved data is represented accurately for a given number of selected delay times.

The average inter-dominance period algorithm 309 then determines which of the selected delay times corresponds to the smallest number of incorrect values and defines this as an average inter-dominance period of the user.

Having determined this average inter-dominance period, in some embodiments the average inter-dominance period algorithm 309 then performs the iterative method a second time. When performing the method for the second time, the new selected delay times are selected from a narrower range centred at the previously determined average inter-dominance period value and are separated by shorter time intervals than the first time. The average inter-dominance period algorithm 309 then determines which of the new selected delay times corresponds to the smallest number of incorrect values and defines this as a second average inter-dominance period of the user. The smaller the time interval between successive selected delay times, the more accurate the determination of the delay time that minimises the number of incorrect dominance switch indicators. Performing the method for a second time in this way therefore improves the accuracy of this determination without requiring the count process to be performed for selected delay times that are far from the previously-calculated average. The second average inter-dominance period of the user will therefore be more accurate than the average inter-dominance period that was first determined.

The average inter-dominance period algorithm 309 calculates the average inter-dominance period confidence using the values of dominance switch indicator confidence retrieved from the dominance switch database 308. This may be achieved using any suitable mathematical calculation for combining two confidence values that is known in the art. However, the present disclosure is not limited to this and in other embodiments the average inter-dominance period algorithm 309 may additionally or alternatively calculate the average inter-dominance period confidence using other factors, such as the number of selected delay times for which a count was performed and the time interval between them.

In additional embodiments of the present disclosure, one or more results based on the parameter related to a psychiatric disorder calculated by the severity assessment algorithm 310 are provided to a recipient. In these embodiments the system 300 further comprises a message system which provides such results to a recipient in the form of messages and a message algorithm that determines properties of the messages and sends information indicating these properties to the message system.

The recipient may be the user, a medical professional, another person or another device. The message system comprises hardware and/or software elements configured to present messages to the recipient in the form of sounds, images, vibrations or the like. The message system may therefore comprise hardware such as a visual display, an audio speaker, a vibration element or the like. In some embodiments the message system comprises components incorporated in a personal device (such as a smartphone, tablet, personal computer or the like) and/or a wearable device (such as earphones, AR glasses, VR goggles or the like). In some embodiments the message system is part of the same device as the display system.

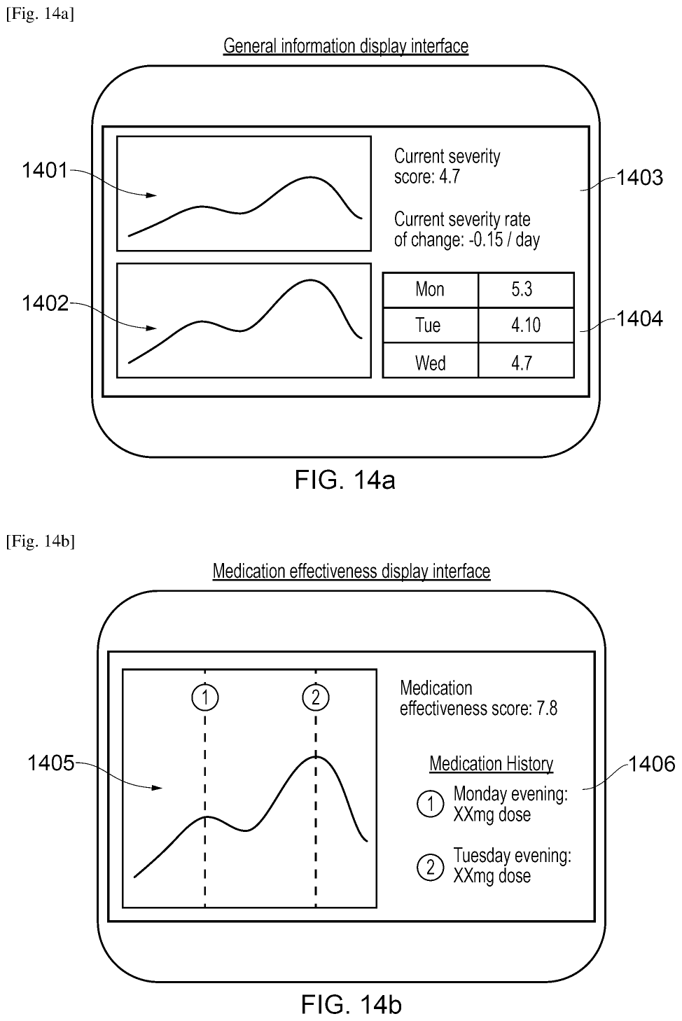

The message algorithm may determine message properties based on preconfigured settings, input from the recipient, message content or the like in various embodiments. Examples of the determination of different message properties in embodiments are described in more detail with reference to FIGS. 14-16.

The message algorithm determines the form taken by a part or entirety of a message (e.g. whether a graph, text, image or sound is included) as well as properties such as the configuration of a graph, the size and colour of text, the size and position of an image, the duration and volume of a sound. In some embodiments the message properties further include the information a message conveys, which the message algorithm determines based on values of the parameter related to a psychiatric disorder and/or other factors (such as the user's medication history). In some embodiments the message algorithm determines when the message system presents messages to the user (e.g. messages appearing on a personal device whenever new message content or properties are determined), but this may also be controlled by the recipient (e.g. the recipient choosing to enter an app that provides messages). In some embodiments, a notification is displayed under certain conditions (e.g. when a severity calculated by the severity assessment algorithm 310 exceeds a certain threshold) that prompts the recipient to select an option that displays more messages.

In embodiments where feedback is provided, methods 600 and 1200 are first completed. In some embodiments the message algorithm then determines message properties based on values of parameter related to of a psychiatric disorder, average inter-dominance period of the user and the associated confidences that were calculated by the average inter-dominance period algorithm 309 and the severity assessment algorithm 310 before sending information indicating these properties to the message system. However, the present disclosure is not limited to this and in other embodiments the message system itself is configured to receive data calculated by the average inter-dominance period algorithm 309 and the severity assessment algorithm 310 and update existing messages automatically. For example, the message system may be configured to add new data points to graphs, new entries to tables and/or update severity scores until no new data is received or until the message algorithm instructs it to stop.

FIGS. 14a and 14b each show messages with various properties displayed on the screen of a personal device. In FIGS. 14a, 1401 and 1402 are graphs showing a rate of change in calculated severity with time and a rate of change in calculated average inter-dominance period respectively. The region 1403 comprises text indicating a value of severity that has been calculated the most recently (the ‘current severity score’) and a rate of change in severity over a certain time period. Some of the text is a colour that indicates a relative classification of the score (e.g. green for scores classed as ‘relatively high’ or red for scores classed as ‘relatively low’). The region 1404 comprises a table of severity scores calculated from data recorded on particular days of the week.

FIG. 14b shows messages with content that was calculated based on additional information. This additional information, the medication history of the user, may have been provided by a doctor or the user. In another embodiment the message algorithm is preconfigured with additional information or receives additional information from another component. 1405 is a graph showing a rate of change in severity over a certain time period in relation to two points in time (indicated by the dotted lines) when the user took doses of medication. The region 1406 comprises text indicating a value of medication effectiveness (the ‘medication effectiveness score’). Region 1406 also comprises text indicating information relating to the two most recent medication doses (the ‘medication history’).

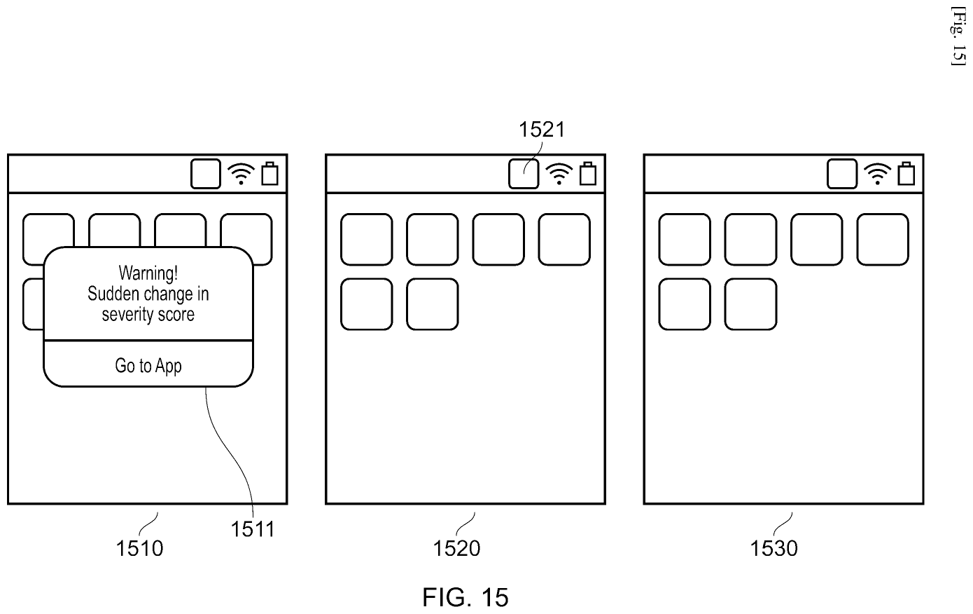

FIG. 15 shows how a notification displayed on a personal device appears in some embodiments when different message properties are determined according to the ‘invasiveness’ of the notification type. 1510 shows a message in the form of a pop-up 1511 that obscures other elements of the user interface. In the present embodiments, this type of message is classed as having ‘high invasiveness’. 1520 shows a message in the form of a notification icon 1521 in a region of the user interface reserved for notifications. This type of message is classed as having ‘medium invasiveness’. 1510 shows a user interface with no visible notification displayed. This type of message is classed as having ‘low invasiveness’. The message algorithm determines the message properties based on a desired level of invasiveness. In some embodiments the desired level of invasiveness is determined based on a severity value (e.g. a severity value calculated using data that was recorded in the most recent series of tests). For example, a notification type with ‘high invasiveness’ may be selected if a severity value exceeds a certain threshold, or a notification type with ‘low invasiveness’ may be selected if a severity value is below a certain threshold. In another example, a notification type with ‘high invasiveness’ may be selected in response to a rapid increase in calculated severity compared to previous values. In another example, a high invasiveness notification may be selected based on external information (e.g. information indicating the user has taken medication within the previous hour), or on both external information and severity (e.g. information indicating that, during an hour immediately following the user taking medication, the rate of change severity did not exceed a certain threshold). In some embodiments, the level of invasiveness may be selected based on the intended recipient (e.g. messages with high invasiveness may not be selected when the intended recipient is a doctor).

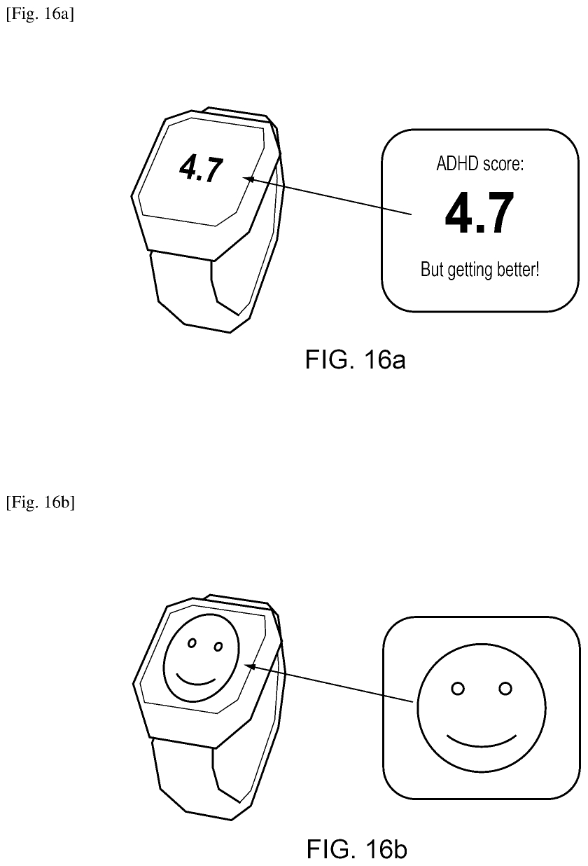

FIGS. 16a and 16b show how messages appear in certain embodiments in which message properties are determined according to the intended recipient of the message. Message properties are categorised by how ‘appropriate’ they are considered to be for a certain recipient. For example, more simplistic messages are considered ‘appropriate for children’ and categorised as such. In FIG. 16a, the message comprises text indicating a severity value (the ‘ADHD score’) and a recent rate of change (the message ‘but getting better!’), as well as a background colour indicating a classification of the severity value (e.g. green for ‘relatively high’ or red for ‘relatively low’). These message properties are considered ‘not appropriate for children’. In FIG. 16b, the message comprises a simplistic depiction of a face with a colour and expression that correspond to a classification of the severity (e.g. green and smiling for ‘relatively high’ or red and frowning for ‘relatively low’). These message properties are considered ‘appropriate for children’. The message algorithm is therefore configured to determine message properties according to the intended recipient, for example selecting the message shown in FIG. 16a if the intended recipient is an adult and selecting the message shown in FIG. 16b if the intended recipient is a child.

In further embodiments the severity assessment algorithm 310 is configured to calculate a parameter related to each of multiple psychiatric disorders in step 1202 of method 1200. Parameter values for different psychiatric disorders are calculated based on different information in various embodiments: for example, in some embodiments a severity of a certain psychiatric disorder may be calculated using an average inter-dominance period value whilst a severity of another disorder may be calculated using a rate of change of average inter-dominance period. However, the present disclosure is not limited to this and in other embodiments the severity assessment algorithm 310 may calculate parameter values for different psychiatric disorders based on the same information. For example, it may be determined that a certain rate of change of average inter-dominance period corresponds to a particular severity of ADHD and another particular severity of OCD. The same test is therefore used to assess the severity of multiple psychiatric disorders simultaneously. In some embodiments the parameter values for different disorders are sent to a message system that provides results to a recipient in the form of messages as described above. This allows the recipient to compare the different parameter values, for example when the values are displayed alongside each other, in order to assist in diagnosis.

In further embodiments the system 300 includes additional components configured to calculate other factors based on data output by the average inter-dominance period algorithm. These components include one or more differentiation algorithms and a disorder probability algorithm. In some embodiments further components such as an inter-dominance period variability algorithm are included. The inter-dominance period variability algorithm determines an average inter-dominance period variability of inter-dominance period values retrieved from the average inter-dominance period algorithm 309. A differentiation algorithm calculates key values to be used in differentiating potential disorders from one another and calculates relative probabilities of the presence of different psychiatric disorders (‘disorder probabilities’) based on the key values.

The disorder probability algorithm calculates total probabilities of the presence of different psychiatric disorders (‘total disorder probabilities’) based on data received from at least one differentiation algorithm. In embodiments where the system 300 includes these components, after step 1201 of method 1200 has been completed the inter-dominance period variability algorithm calculates an inter-dominance period variability based on average inter-dominance period values and the differentiation algorithms are run.

In some embodiments the differentiation algorithms include a variance-mode algorithm and a bipolar disorder differentiation algorithm. The variance-mode algorithm receives an inter-dominance period variability from the inter-dominance period variability algorithm and average inter-dominance period values from the average inter-dominance period algorithm then determines a two-dimensional vector (the ‘variance-mode vector’) that consists of the inter-dominance period variability and a calculated modal value of retrieved inter-dominance period values. The variance-mode algorithm then calculates the magnitude of the difference between the calculated variance-mode vector and each of a set of characteristic variance-mode vectors associated with different disorders. The algorithm generates a disorder probability for each disorder based on each calculated magnitude. The bipolar disorder differentiation algorithm analyses a change in inter-dominance period value over time and then determines an average periodicity and range of inter-dominance period values with associated confidence values. This may be achieved using any suitable statistical analysis method that is known in the art. The average periodicity and range are then compared with typical values for the change in inter-dominance period between different phases of bipolar disorder in a user. The algorithm generates a disorder probability for bipolar disorder based on the results of the comparison.

The disorder probability algorithm then calculates total disorder probabilities based on the outputs of the differentiation algorithms. For example, the disorder probability algorithm may calculate a total disorder probability of the presence of bipolar disorder in the user based on disorder probabilities output by a variance-mode algorithm and a bipolar disorder differentiation algorithm.

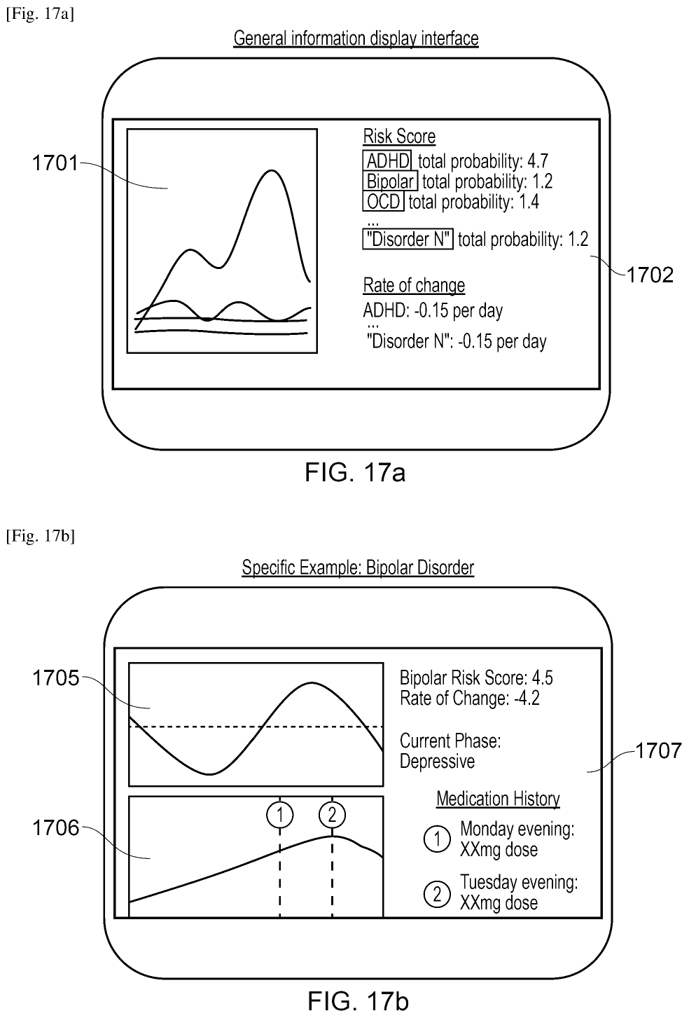

In some embodiments the total disorder probabilities are output to a message system such as those described above in relation to FIGS. 14-16. FIGS. 17a and 17b depict messages presented to a recipient in embodiments. In FIG. 17a, 1701 is a graph showing total disorder probabilities of a number of disorders over time. The region 1702 comprises text indicating a total disorder probability for each of the disorders and a rate of change of each total disorder probability at a particular time. In FIG. 17b) 1705 is a graph showing the average inter-dominance period over time, divided into two sections indicating which phase of the disorder the user was experiencing at a particular time. 1706 is a graph showing the total disorder probability of bipolar disorder over time in relation to two points in time (indicated by the dotted lines) when the user took doses of medication. The region 1707 comprises text indicating the current total disorder probability of bipolar disorder and its rate of change, as well as the current phase of the disorder the user is experiencing. Region 1707 also comprises text indicating information relating to the two most recent medication doses (the ‘medication history’).

In further embodiments a parameter related to a psychiatric disorder is calculated by the severity assessment algorithm 310 based on a determined user reaction time. The user reaction time is defined as the time interval between when the display system 304 first starts to display the test feature to the user and when the user begins to produce a user response. In these embodiments, an alternate method (method 1800) to methods 600 and 1200 is carried out in order to determine a parameter related to a psychiatric disorder. In some embodiments method 1800 is carried out alongside methods 600 and 1200.

Method 1800 is carried out by the system 300, with the addition of a user response reaction time algorithm, a reaction time database and an average reaction time algorithm. In some embodiments a combined severity assessment algorithm is also included. The user response reaction time algorithm calculates a user reaction time and an associated confidence (the user reaction time confidence). The reaction time database stores the calculated reaction times and associated confidence values. The average reaction time algorithm calculates an average reaction time and associated confidence based on the data stored in the reaction time database, and the combined severity assessment algorithm determines a combined parameter related to a psychiatric disorder (for example a combined severity of the psychiatric disorder). In some embodiments the severity assessment algorithm is additionally or alternatively configured to calculate a parameter related to a psychiatric disorder based on an average user reaction time.

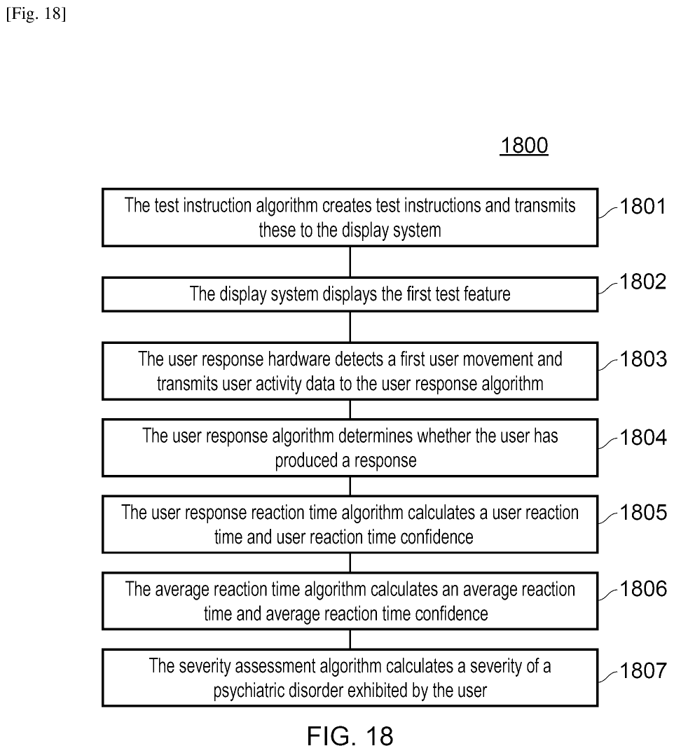

FIG. 18 is a flow chart of a method 1800. In step 1801 the test instruction algorithm 302 creates test instructions and transmits these to the display system 304. This step is identical to step 602 of method 600 except that the test instructions comprise only the test feature ID of a first test feature to be displayed to the user and one or more visual feature IDs associated with this first test feature. The test instructions do not comprise an order in which two test features are to be displayed to the user or a determined delay time. In step 1802 the display system 304 displays the first test feature to the user according to the test instructions in the same way as in step 603. In step 1803 the user response hardware 305 detects a first user movement and transmits user activity data to the user response algorithm 306. This step is identical to step 604 except that the user activity data always comprises time information indicating the time over which the first user movement occurred in relation to when the display system 304 displayed the first test feature to the user. In step 1804 the user response algorithm 306 determines whether the user has produced a predetermined response. This step is the same as step 605. In step 1805 the user response reaction time algorithm receives a user response A and user response A confidence value from the user response algorithm 306 and the user activity data from the user response hardware 305. The user response reaction time algorithm then calculates a user reaction time and associated user reaction time confidence based on the retrieved data. In some embodiments the user response reaction time algorithm calculates a user reaction time and associated user reaction time confidence if the user response A indicates that a user response was produced. In other embodiments the user response reaction time algorithm calculates a user reaction time and associated confidence if the user response A indicates that a user response was produced and the user response A confidence value exceeds a certain threshold.

In some embodiments the user reaction time is defined as the time interval between when the display system 304 first starts to display the test feature to the user and when the user first starts to move a joystick in a particular direction. The present disclosure is not limited to this however and the user reaction time may be calculated in any suitable manner, depending on the nature of the user response. For example, in other embodiments the user reaction time is the time interval between when the display system 304 starts to display the test feature and when the user's eyes are moved to look at a fixation location. The user reaction time confidence may also be calculated in any suitable manner depending on the nature of the user response.

The user response reaction time algorithm transmits the calculated user reaction time and associated confidence to the reaction time database to be stored. In some embodiments, before step 1806 is carried out a data sufficiency check algorithm determines whether a number of data entries in the reaction time database has reached a predetermined sufficiency threshold. Such a data sufficiency check algorithm could be configured in the same way as the data sufficiency check algorithm described earlier following method 600. In step 1806 the average reaction time algorithm retrieves this data and calculates an average reaction time and average reaction time confidence based on one or more values of calculated user reaction time and user reaction time confidence. In a similarly way to the average inter-dominance period algorithm 309, in some embodiments the average reaction time algorithm will only retrieve certain data entries to calculate this average.

In step 1807 the severity assessment algorithm calculates a parameter related to a psychiatric disorder and associated confidence based on an average user reaction time and associated confidence. This is achieved in a number of different ways in various embodiments. For example, a calculated severity may take one of two values (indicating ‘at risk’ and ‘not at risk’ respectively) depending on whether the average reaction time exceeds a particular threshold. In another example, a calculated severity may take a number of discrete values between 1 and 0 based on a categorization of the average user reaction time (e.g. an average user reaction time may correspond to a severity value of 0.2).

In some embodiments in which method 1800 is carried out alongside methods 600 and 1200, the severity assessment algorithm calculates both a parameter (the reaction time parameter) and associated confidence based on an average user reaction time and associated confidence and a parameter (the inter-dominance period parameter) and associated confidence based on an average inter-dominance period and associated confidence. In some of these embodiments a combined severity assessment algorithm then retrieves the reaction time parameter, the inter-dominance period parameter and the associated confidence values and then calculates a combined parameter related to the psychiatric disorder and associated confidence based on the retrieved data using any suitable means. The weighting of each of the two parameter values in this calculation may be equal, or based on other factors. For example, in some embodiments the weighting is based on the confidence values associated with the parameter. In other embodiments the weighting is additionally or alternatively based on a predetermined condition (e.g. an inter-dominance severity may be given a default precedence).

FIG. 19 is a diagram of a system 1900 in which the components of the system 300 are located in device 1901 and server 1902. Device 1901 is a user device comprising the test feature database 301, the test instruction algorithm 302, the delay time definition algorithm 303, the display system 304, the user response hardware 305 and the user response algorithm 306. Server 1902 is a server comprising the dominance switch detection algorithm 307, the dominance switch database 308, the average inter-dominance period algorithm 309 and the severity assessment algorithm 310.

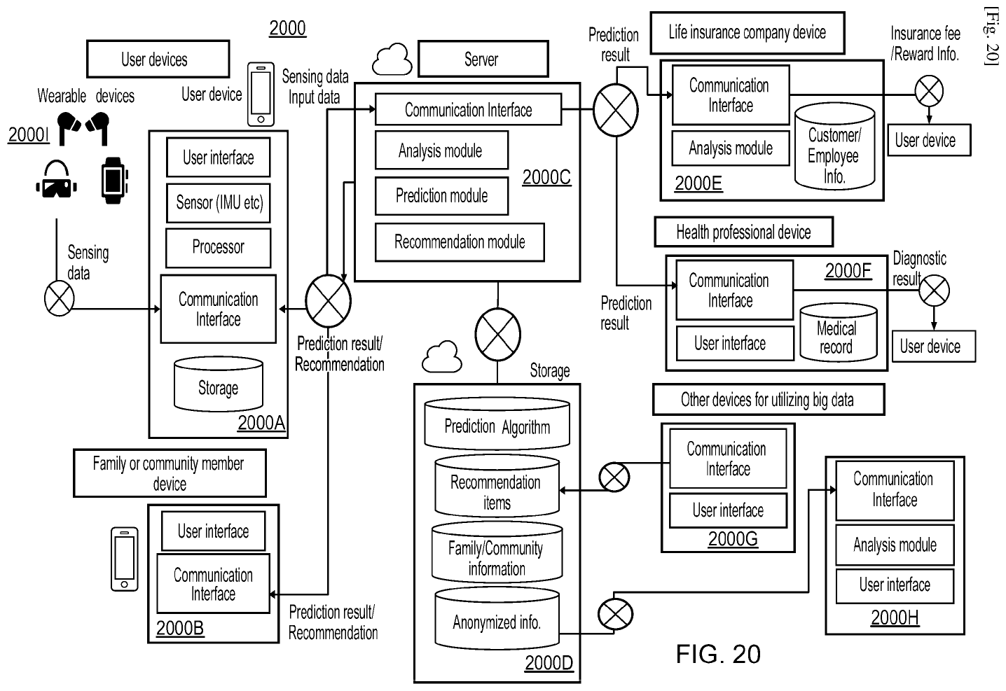

Although the foregoing has been described with reference to embodiments being carried out on a device or various devices that are components of the system 300, the disclosure is not so limited. In embodiments, the disclosure may be carried out on a system 2000 such as that shown in FIG. 20.

In the system 2000, the wearable devices 2000I are devices that are worn on a user's body. For example, the wearable devices may be earphones, a smart watch, Virtual Reality Headset or the like. The wearable devices contain sensors that measure the movement of the user and which create sensing data to define the movement or position of the user. This sensing data is provided over a wired or wireless connection to a user device 2000A. Of course, the disclosure is not so limited. In embodiments, the sensing data may be provided directly over an internet connection to a remote device such as a server 2000C located on the cloud. In further embodiments, the sensing data may be provided to the user device 2000A and the user device 2000A may provide this sensing data to the server 2000C after processing the sensing data. In the embodiments shown in FIG. 20, the sensing data is provided to a communication interface within the user device 2000A. The communication interface may communicate with the wearable device(s) using a wireless protocol such as low power Bluetooth or WiFi or the like.

The user device 2000A is, in embodiments, a mobile phone or tablet computer. The user device 2000A has a user interface which displays information and icons to the user. Within the user device 2000A are various sensors such as gyroscopes and accelerometers that measure the position and movement of a user. The operation of the user device 2000A is controlled by a processor which itself is controlled by computer software that is stored on storage. Other user specific information such as profile information is stored within the storage for use within the user device 2000A. As noted above, the user device 2000A also includes a communication interface that is configured to, in embodiments, communicate with the wearable devices. Moreover, the communication interface is configured to communicate with the server 2000C over a network such as the Internet. In embodiments, the user device 2000A is also configured to communicate with a further device 2000B. This further device 2000B may be owned or operated by a family member or a community member such as a carer for the user or a medical practitioner or the like. This is especially the case where the user device 2000A is configured to provide a prediction result and/or recommendation for the user. The disclosure is not so limited and in embodiments, the prediction result and/or recommendation for the user may be provided by the server 2000C.

The further device 2000B has a user interface that allows the family member or the community member to view the information or icons. In embodiments, this user interface may provide information relating to the user of the user device 2000B such as diagnosis, recommendation information or a prediction result for the user. This information relating to the user of the user device 2000B is provided to the further device 2000B via the communication interface and is provided in embodiments from the server 2000C or the user device 2000A or a combination of the server 2000C and the user device 2000A.

The user device 2000A and/or the further device 2000B are connected to the server 2000C. In particular, the user device 2000A and/or the further device 2000B are connected to a communication interface within the server 2000C. The sensing data provided from the wearable devices and or the user device 2000A are provided to the server 2000C. Other input data such as user information or demographic data is also provided to the server 2000C. The sensing data is, in embodiments, provided to an analysis module which analyses the sensing data and/or the input data. This analysed sensing data is provided to a prediction module that predicts the likelihood of the user of the user device having a condition now or in the future and in some instances, the severity of the condition. The predicted likelihood is provided to a recommendation module that provides a recommendation to the user and/or the family or community member. Although the prediction module is described as providing the predicted likelihood to the recommendation module, the disclosure is not so limited and the predicted likelihood may be provided directly to the user device 2000A and/or the further device 2000B.

Additionally, connected to or in communication with the server 2000C is storage 2000D. The storage 2000D provides the prediction algorithm that is used by the prediction module within the server 2000C to generate the predicted likelihood. Moreover, the storage 2000D includes recommendation items that are used by the recommendation module to generate the recommendation to the user. The storage 2000D also includes in embodiments family and/or community information. The family and/or community information provides information pertaining to the family and/or community member such as contact information for the further device 2000B.

Also provided in the storage 2000D is an anonymised information algorithm that anonymises the sensing data. This ensures that any sensitive data associated with the user of the user device 2000A is anonymised for security. The anonymised sensing data is provided to one or more other devices which is exemplified in FIG. 20 by device 2000H. This anonymised data is sent to the other device 2000H via a communication interface located within the other device 2000H. The anonymised data is analysed with the other data 2000H by an analysis module to determine any patterns from a large number set of sensing data. This analysis will improve the recommendations made by the recommendations module and will improve the predictions made from the sensing data. Similarly, a second other device 2000G is provided that communicates with the storage 2000D using a communication interface.

Returning now to server 2000C, as noted above, the prediction result and/or the recommendation generated by the server 2000C is sent to the user device 2000A and/or the further device 2000B.

Although the prediction result is used in embodiments to assist the user or his or her family member or community member, the prediction result may be also used to provide more accurate health assessments for the user. This will assist in purchasing products such as life or health insurance or will assist a health professional. This will now be explained.

The prediction result generated by server 2000C is sent to the life insurance company device 2000E and/or a health professional device 2000F. The prediction result is passed to a communication interface provided in the life insurance company device 2000E and/or a communication interface provided in the health professional device 2000F. In the event that the prediction result is sent to the life insurance company device 2000E, an analysis module is used in conjunction with the customer information such as demographic information to establish an appropriate premium for the user. In instances, rather than a life insurance company, the device 2000E could be a company's human resources department and the prediction result may be used to assess the health of the employee. In this case, the analysis module may be used to provide a reward to the employee if they achieve certain health parameters. For example, if the user has a lower prediction of ill health, they may receive a financial bonus. This reward incentivises healthy living. Information relating to the insurance premium or the reward is passed to the user device.

In the event that the prediction result is passed to the health professional device 2000F, a communication interface within the health professional device 2000F receives the prediction result. The prediction result is compared with the medical record of the user stored within the health professional device 2000F and a diagnostic result is generated. The diagnostic result provides the user with a diagnosis of a medical condition determined based on the user's medical record and the diagnostic result is sent to the user device.

In the system 2000, in embodiments the wearable devices 2000I serve the same functions as the display system 304 and/or the user response hardware 305 in the system 300 described earlier. In embodiments in which the user device 2000A is used in the collection of user activity data, the user device 2000A similarly serves these functions. Components which are functionally equivalent to the test feature database 301, the test instruction algorithm 302 and the delay time definition algorithm 303 may each be found in any of the wearable devices 20001, the user device 2000A and the server 2000C. In some embodiments the analysis module in the server 2000C comprises components that are functionally equivalent to the user response algorithm 306, the dominance switch detection algorithm 307 and the average inter-dominance period algorithm 309 when carrying out the present disclosure. Either the prediction module in server 2000C or, in some embodiments, the storage 2000D, is configured to perform the same processes as the severity assessment algorithm 310 described above. Whilst examples are given here in which certain components of the system 2000 perform the same function as the previously-described components of the system 300, the disclosure is not so limited. In other embodiments, the method according to the present disclosure may be performed by the various components of the system 2000 in any suitable manner in place of the system 300.

Numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

Embodiments of the disclosure are provided in the following numbered clauses:

(1)

A method for generating a parameter related to a psychiatric disorder comprising: displaying to a user a first image set, the first image set comprising a first image having a first trigger image and a second image different from the first image, such that the first image and the second image are displayed to each eye of the user;
detecting a first movement of the user whilst the first image set is displayed;
displaying to the user a second image set, the second image set comprising a third image having a second trigger image and a fourth image different from the third image, such that the third image and the fourth image are displayed to each eye of the user;
detecting a second movement of the user whilst the second image set is displayed;
and
generating a parameter related to the psychiatric disorder in the user based on user activity data that is indicative of the first movement of the user and the second movement of the user.
   (2)

A method according to clause (1), wherein the fourth image is displayed on the same display area as the first image and the third image is displayed on the same display area as the second image.

(3)

A method according to any preceding clause, wherein the first image and the second image depict the same subject from different angles.

(4)

A method according to any preceding clause, wherein the parameter related to the psychiatric disorder in the user is based on the average inter-dominance period of the user's eyes calculated from the user activity data that is indicative of the first movement of the user and the second movement of the user.

(5)

A method according to any preceding clause, wherein the parameter related to the psychiatric disorder in the user is based on a user response confidence calculated from user activity data that is indicative of the first movement of the user and the second movement of the user.

(6)

A method according to any preceding clause, wherein the first trigger image and the second trigger image are configured to incite a predetermined response in a user.

(7)

A method according to any preceding clause, wherein the psychiatric disorder is attention deficit hyperactivity disorder.

(8)

A method according to any preceding clause, wherein at least one of the first movement of the user and the second movement of the user is an eye movement.

(9)

A method according to any preceding clause, wherein the method further includes generating a parameter related to a second psychiatric disorder in the user based on user activity data that is indicative of the first movement of the user and the second movement of the user.

(10)

A method according to any preceding clause, wherein the method further comprises notifying a result that indicates the risk of the psychiatric disorder in the user measured by the parameter.

(11)

A method according to clause (10), wherein the result further indicates a change in the risk of the psychiatric disorder in the user over time.

(12)

A method according to any preceding clause, wherein the method further comprises notifying a result that indicates the severity of the psychiatric disorder in the user measured by the parameter.

(13)

A method according to any preceding clause, wherein the method further comprises notifying a result that indicates a medication effectiveness in the user measured by the parameter.

(14)

A method according to clause (1), wherein the first image set and the second image set depict a user interface scene, and the first trigger image and the second trigger image indicate a notification for the user.

(15)

A method according to clause (1), wherein the first image set and the second image set depict a video game scene; and the first trigger image and the second trigger image include a target object for a player to attempt to reach or an object for the player to avoid.

(16)

A method for generating a parameter related to a psychiatric disorder comprising:
displaying to a user an image set, the image set comprising two different images that include a trigger image configured to incite a response in the user, such that the two different images are displayed to each eye of the user;
detecting a movement of the user whilst the image set is displayed;
calculating the user's average reaction time when responding to the trigger image; and
generating a parameter related to the psychiatric disorder in the user based on the user's average reaction time when responding to the trigger image.
   (17)

A system (1900) for generating a parameter related to a psychiatric disorder comprising:
a display system (304) configured to display to a user a first image set, the first image set comprising a first image having a first trigger image and a second image different from the first image, such that the first image and the second image are displayed to each eye of the user; then display to the user a second image set, the second image set comprising a third image having a second trigger image and a fourth image different from the third image, such that the third image and the fourth image are displayed to each eye of the user;
user response hardware (305) configured to detect a first movement of the user whilst the first image set is displayed and detect a second movement of the user whilst the second image set is displayed; and
a severity assessment algorithm (310) configured to generate a parameter related to the psychiatric disorder in the user based on user activity data that is indicative of the first movement of the user and the second movement of the user.
   (18)

A user device (1901) configured to display to a user a first image set, the first image set comprising:
a first image having a first trigger image and a second image different from the first image, such that the first image and the second image are displayed to each eye of the user;
detect a first movement of the user whilst the first image set is displayed; display to the user a second image set, the second image set comprising a third image having a second trigger image and a fourth image different from the third image, such that the third image and the fourth image are displayed to each eye of the user;
   detect a second movement of the user whilst the second image set is displayed; and
   produce user activity data that is indicative of the first movement of the user and the second movement of the user such that a parameter related to the psychiatric disorder in the user may be generated based on the user activity data.
      (19)

A computer program comprising computer readable software, which when loaded onto a computer configures the computer to perform a method according to clause (1).

1. A method for generating a parameter related to a psychiatric disorder comprising:
displaying to a user a first image set, the first image set comprising a first image having a first trigger image and a second image different from the first image, such that the first image and the second image are displayed to each eye of the user;
detecting a first movement of the user whilst the first image set is displayed;
displaying to the user a second image set, the second image set comprising a third image having a second trigger image and a fourth image different from the third image, such that the third image and the fourth image are displayed to each eye of the user;
detecting a second movement of the user whilst the second image set is displayed;
and
generating a parameter related to the psychiatric disorder in the user based on user activity data that is indicative of the first movement of the user and the second movement of the user.

2. A method according to claim 1, wherein the fourth image is displayed on the same display area as the first image and the third image is displayed on the same display area as the second image.

3. A method according to claim 2, wherein the first image and the second image depict the same subject from different angles.

4. A method according to claim 1, wherein the parameter related to the psychiatric disorder in the user is based on the average inter-dominance period of the user's eyes calculated from the user activity data that is indicative of the first movement of the user and the second movement of the user.

5. A method according to claim 1, wherein the parameter related to the psychiatric disorder in the user is based on a user response confidence calculated from user activity data that is indicative of the first movement of the user and the second movement of the user.

6. A method according to claim 1, wherein the first trigger image and the second trigger image are configured to incite a predetermined response in a user.

7. A method according to claim 1, wherein the psychiatric disorder is attention deficit hyperactivity disorder.

8. A method according to claim 1, wherein at least one of the first movement of the user and the second movement of the user is an eye movement.

9. A method according to claim 1, wherein the method further includes generating a parameter related to a second psychiatric disorder in the user based on user activity data that is indicative of the first movement of the user and the second movement of the user.

10. A method according to claim 1, wherein the method further comprises notifying a result that indicates the risk of the psychiatric disorder in the user measured by the parameter.

11. A method according to claim 10, wherein the result further indicates a change in the risk of the psychiatric disorder in the user over time.

12. A method according to claim 1, wherein the method further comprises notifying a result that indicates the severity of the psychiatric disorder in the user measured by the parameter.

13. A method according to claim 1, wherein the method further comprises notifying a result that indicates a medication effectiveness in the user measured by the parameter.

14. A method according to claim 1, wherein the first image set and the second image set depict a user interface scene, and the first trigger image and the second trigger image indicate a notification for the user.

15. A method according to claim 1, wherein the first image set and the second image set depict a video game scene; and the first trigger image and the second trigger image include a target object for a player to attempt to reach or an object for the player to avoid.

16. A method for generating a parameter related to a psychiatric disorder comprising:
displaying to a user an image set, the image set comprising two different images that include a trigger image configured to incite a response in the user, such that the two different images are displayed to each eye of the user;
detecting a movement of the user whilst the image set is displayed;
calculating the user's average reaction time when responding to the trigger image; and
generating a parameter related to the psychiatric disorder in the user based on the user's average reaction time when responding to the trigger image.

17. A system for generating a parameter related to a psychiatric disorder comprising:
a display system configured to display to a user a first image set, the first image set comprising a first image having a first trigger image and a second image different from the first image, such that the first image and the second image are displayed to each eye of the user; then display to the user a second image set, the second image set comprising a third image having a second trigger image and a fourth image different from the third image, such that the third image and the fourth image are displayed to each eye of the user;
user response hardware configured to detect a first movement of the user whilst the first image set is displayed and detect a second movement of the user whilst the second image set is displayed; and
a severity assessment algorithm configured to generate a parameter related to the psychiatric disorder in the user based on user activity data that is indicative of the first movement of the user and the second movement of the user.

18. A user device configured to display to a user a first image set, the first image set comprising:
a first image having a first trigger image and a second image different from the first image, such that the first image and the second image are displayed to each eye of the user;
detect a first movement of the user whilst the first image set is displayed; display to the user a second image set, the second image set comprising a third image having a second trigger image and a fourth image different from the third image, such that the third image and the fourth image are displayed to each eye of the user;
detect a second movement of the user whilst the second image set is displayed; and
produce user activity data that is indicative of the first movement of the user and the second movement of the user such that a parameter related to the psychiatric disorder in the user may be generated based on the user activity data.

19. A computer program comprising computer readable software, which when loaded onto a computer configures the computer to perform a method according to claim 1.
